(19) United States
(12) Patent Application Publication
(10) Pub. No.: US 2023/0060850 A1
(43) Pub. Date: Mar. 2, 2023

(54) DEVICE FOR REDUCTION OR PREVENTION OF PRETERM LABOR

(71) Applicant: Novocuff, Inc., San Luis Obispo, CA (US)

(72) Inventors: Donald J. Lee, San Luis Obispo, CA (US); Amelia M. Degenkolb, Birmingham, AL (US)

(21) Appl. No.: 17/813,454

(22) Filed: Jul. 19, 2022

Related U.S. Application Data
(60) Provisional application No. 63/300,263, filed on Jan. 18, 2022.
(60) Provisional application No. 63/237,708, filed on Aug. 27, 2021.

(51) Int. Cl. A61B 17/42; A61F 6/08; A61B 17/12

(52) U.S. Cl. CPC A61B 17/42; A61F 6/08; A61B 17/1204; A61B 17/12099; A61B 17/12136; A61B 2017/4225

(57) ABSTRACT

A medical device for supporting a patient's cervix, such as in the event of an insufficient cervix or uterine prolapse. The device includes a main body configured for placement in a vagina, a proximal opening at an upper portion of the body, and a distal opening at a lower portion of the body. A portion of the body engages and anchors the device along the vaginal wall proximate the cervical opening. An expandable cuff at the proximal opening is expandable to circumscribe the exterior of the cervix. The cuff is expandable inward toward the cervix, or downward away from the uterus, or both inward and downward. The cuff may include multiple features, such as a recessed portion and an enlargeable portion arranged adjacent one another, in which the recessed portion may permit blood flow to lower cervical tissue.

### CROSS REFERENCE TO RELATED APPLICATIONS

The present application claims priority of U.S. provisional application Ser. No. 63/237,708 filed Aug. 27, 2021, and Ser. No. 63/300,263, filed Jan. 18, 2022, which are each hereby incorporated herein by reference in their entireties.

### FIELD OF THE INVENTION

The present invention is directed to reproductive, gestational, and prenatal health, and in particular to devices for reduction or prevention of preterm labor.

### BACKGROUND OF THE INVENTION

The most common reason for infant mortality is complications due to preterm labor. Several factors contribute to early births, including most commonly, cervical insufficiency, otherwise referred to medically as a “short cervix”. When a short cervix occurs in singleton pregnancies (i.e. single fetus pregnancies), a common intervention method includes a suture(s) placed in the cervix in attempts to delay birth, known as a cervical cerclage. Generally, interventions for cervical insufficiency in multiple gestations (i.e. multiple fetus pregnancies) are limited and may provide only limited efficacy. When placed in a patient, some commonly known or commercially available cervical support devices constrict or block the flow of blood/bodily fluid to the lower extremities of the cervix.

### SUMMARY OF THE INVENTION

The present invention provides a device that reinforces and supports the cervix to promote an extended gestation period in patients experiencing complications from preterm labor due to an insufficient cervix. The device is referred to herein as a cervical support device. The device provides selectively adjustable or customizable support for a patient's cervix and may aid in delaying labor with both singleton and multiple gestation pregnancies in individuals experiencing an insufficient cervix. In some arrangements, the device may be utilized to support or reduce the effects of pelvic organ prolapse. The device includes one or more inflatable cuffs or bladders, which are selectively inflatable and deflatable to provide a desired amount of support and/or position correction to the cervix. The cuffs may be independently inflatable, or may be collectively inflatable. The device may include an upper or proximal cuff to engage and support the cervix, uterus, and/or fetus, and an outer and/or lower distal cuff to engage the patient's vaginal wall to support the device in proximity to the cervix. The proximal cuff may be utilized to provide a constricting, compressive force to the exterior of the lower portion of the cervix, in an attempt to retain the cervix in a closed state. The device's cuffs may include one or more individually addressable (i.e. inflatable/deflatable) bladders that cooperate with one another to provide the desired support. The proximal cuff may be configured to permit blood/fluid perfusion in the cervix (such as by inflating expandable portions of the cuff), while providing a sufficient amount of compressive pressure to the cervix. The anatomy of the cervix may vary in its nominal external dimension from patient to patient, and an insufficient cervix may (and is likely to) continue to shrink as the gestation progresses. As such, the adjustable functionality of the cervical support device of the present invention may permit a care provider (i.e. medical provider such as a physician, midwife, or nurse) to customize the support to a particular patient's cervix by directing the inflation/deflation of the cuff(s). The distal cuff may be utilized to adjust the position of the device within the patient's vagina, such as to raise the device relative to the cervix or to redirect the cervix, for example. Optionally, the device may be inverted such that the distal opening faces towards the cervix such that the device may provide support to the pelvic organs (e.g. bladder and uterus) (without directly contacting the lower portion of the cervix) in cases of prolapse (e.g. uterine or cervical prolapse). The device may also be beneficial in reducing or limiting the risk of infection and/or extending fetal development in instances of premature rupture of membranes (PROM) and preterm premature rupture of membranes (PPROM).

According to one form of the present invention, a cervical support and reinforcement device is provided for supporting a patient's cervix and includes a main body dimensioned and shaped for placement in the patient's vagina. The main body defines an interior passageway having a proximal opening at an upper portion of the body and a distal opening at a lower portion of the body. A portion of the exterior perimeter of the body is provided for engaging and pressing or partially embedding into the vaginal wall to anchor the device near the cervical opening. An expandable annular cuff is provided around an interior of the body proximate the proximal opening such that the cuff defines at least a portion of the proximal opening. The cuff is expandable toward the axial center of the proximal opening to circumscribe an exterior portion of the lower cervix. The cuff is expandable from a neutral state to an expanded state, which decreases the area of the proximal opening. In other words, when inflated, the cuff expands inward to constrict the exterior of the cervix. The cuff may include one or more recessed or minimally inflatable portions and one or more enlarged or enlargeable portions that are adjacent the recessed portion(s). The enlarged portions are expandable toward the axial center of the proximal opening to constrict the cervix and the recessed portion of the cuff remains generally uninflated or unexpanded to permit blood flow from the upper cervical tissue to the lower cervical tissue. Optionally, the cervical support device may include an annular ridge along the interior perimeter of the cuff for gripping or engaging the exterior of the cervix, such as providing improved grip around the cervix as the cuff is inflated.

In one aspect, the main body of the device is a semi-spheroid body formed with a semi-spheroid shape defining a proximal opening at an upper portion of the semi-spheroid body and a distal opening at a lower portion of the semi-spheroid body. The proximal opening has a smaller area than the distal opening. The exterior portion of the body at or near the distal opening may be dimensioned and shaped for engaging the vaginal wall to support the device proximate the cervical opening.

In another aspect, the cuff is expandable radially inward toward the axial center of the proximal opening, as well as downward away from the transverse plane of the proximal opening and away from the uterus as the cuff expands around the cervix. The inward, radial expansion of the proximal cuff acts to constrict an exterior portion of the cervix, while the downward expansion acts to pull or push the cervix downward away from the uterus.

In yet another aspect, the cuff includes two or more adjacent bladders that are positioned, shaped, and dimensioned in a manner such that, upon inflation of one or more of the inflatable bladders, the cuff expands (i) radially inward and/or downward away from the transverse plane of the proximal opening. For example, the cuff may include a first inflatable bladder that is coaxial with the proximal opening and a second inflatable bladder that is coaxial with the first inflatable bladder and the proximal opening. Preferably, the cuff is expandable from a neutral state to an expanded state in which some portion of the cuff is expanded both radially inward toward the axial center of the proximal opening, as well as downward away from the transverse plane of the proximal opening and away from the uterus, to thereby apply a downward force to the exterior of the cervix.

In still another aspect, a radial cross-section of the cuff (e.g. the enlarged portion of the cuff) is formed with a non-uniform wall thickness, such that, upon expansion, the arrangement of the non-uniform wall cuff causes the cuff to expand both radially inward, as well as downward away from the transverse plane of the proximal opening.

In yet another aspect, the cervical support device includes an annular rim at the upper portion of the main body. The annular rim extends upward away from the transverse plane of the proximal opening. Upon placement of the device in the patient's vagina, the annular rim initially receives the lower end of the cervix and then guides the cervix into the proximal opening, which may aid a provider is properly positioning the device proximate the cervix.

A fluid conduit may be disposed in the main body of the cervical support device with a fluid connection to the cuff for the introduction or removal of fluid (e.g. liquid or air) to the cuff. A valve may be provided in fluid communication with the fluid conduit at an opposite end from the cuff. The valve may removably receive a portion of an inflation device (e.g. a pump) that is operable to inflate the cuff without removal of the device from the patient's vagina. The valve may include check valves, such as duckbill valves, to minimize backflow of fluid after it is directed into the cuff.

In still another aspect, the cervical support device includes another or second expandable annular cuff near the lower portion and/or outer (convex) curvature of the main body, such as proximate the distal opening. This additional cuff is referred to hereinafter as the “distal cuff”, whereas the previously discussed cuff is referred to hereinafter as the “proximal cuff”, where applicable. The distal cuff is outwardly expandable, away from the axial center of the distal opening such that it expands toward and into contact with the vaginal wall. The distal cuff is expandable from a neutral state to an expanded state in which the exterior perimeter of the distal cuff is increased (as compared to its exterior perimeter when in the neutral state), to expand into contact with the vaginal wall and to anchor the device at the vaginal wall. The additional or distal cuff may be in fluid communication with a fluid conduit, such as that described above, in order to receive or release fluid to/from the distal cuff. A single fluid conduit may be provided in communication with each cuff of the device, or dedicated fluid conduits may be provided for each cuff, and/or each individually addressable bladder of the cuffs.

In another form of the present invention, a cervical support and reinforcement device is provided for supporting a patient's cervix and includes a main body dimensioned and shaped for placement in the patient's vagina. The main body has an upper portion defining a proximal opening and a lower portion defining distal opening. An exterior portion of the body is configured for engaging the vaginal wall to support or anchor the device proximate the cervical opening. The device includes an expandable annular cuff at an interior of the body, near the proximal opening, and the cuff defines at least a portion of the proximal opening. At least a portion of the cuff is expandable from a neutral state to an expanded state in which the cuff is expanded both (i) radially inward, toward the axial center of the proximal opening to constrict an exterior portion of the cervix, and (ii) downward away from the transverse plane of the proximal opening and away from the uterus to apply a downward force to the exterior of the cervix.

In one aspect, the cuff includes one or more recessed portions and one or more enlarged portions in spaced arrangement with one another. The enlarged portions are expandable toward the axial center of the proximal opening, as well as downward away from the transverse plane of the proximal opening. The recessed portion(s) of the cuff may permit blood flow from the upper cervical tissue to the lower cervical tissue that extends through the cuff.

In another aspect, a radial cross-section of the cuff has a non-uniform wall thickness, such that based on the material characteristics of the non-uniform wall thickness, upon expansion, the cuff is expandable both radially inward and downward away from the transverse plane of the proximal opening.

In yet another aspect, the cuff includes a plurality of inflatable bladders adjacent and co-axial with one another. The plurality of inflatable bladders are arranged such that, upon inflation of one or more of the bladders, the cuff expands radially inward, toward the axial center of the proximal opening and/or downward away from the transverse plane of the proximal opening.

In another aspect, the cervical support device includes a distal expandable annular cuff disposed at the lower portion of the body proximate the distal opening. The distal cuff is outwardly expandable, away from the axial center of the distal opening, to expand toward and into contact with the vaginal wall to anchor the device securely within the vagina.

Accordingly, the cervical support and reinforcement device of the present invention provides a customizable and adjustable device for supporting a cervix of a patient experiencing an insufficient cervix that may progress to a pre-term labor. The device includes one or more inflatable cuffs and/or individually addressable inflatable bladders the permit a care provider to direct a compressive force at desired locations around the lower portion of a patient's cervix. The adjustable nature of the device permits a provider to adjust the amount of pressure applied to the cervix or adjust the orientation of the device relative to the cervix and vagina, all without removal or direct, manual manipulation of the device through the vagina. The cuffs may include various dimensional features or structural aspects that cause the cuff to expand both radially inward toward the cervix, as well as downward away from the uterus to apply an oblique, inward downward force to the cervix.

These and other objects, advantages, purposes, and features of this invention will become apparent upon review of the following specification in conjunction with the drawings.

### BRIEF DESCRIPTION OF THE DRAWINGS

FIG. 1 is a sagittal view of a patient with multiple gestations, illustrating the placement of a cervical support and reinforcement device of the present invention;

FIG. 2 is another sagittal view of the patient of FIG. 1, with the cervical support device shown in a sectional side view with a top, inner cuff in an inflated or expanded state;

FIG. 3 is a top perspective view of the cervical support device of FIG. 1, depicted with a top, inner cuff in a deflated or neutral state;

FIG. 4 is another top perspective view of the cervical support device of FIG. 1, depicted with the top, inner cuff in the inflated state;

FIG. 5 is a sectional side elevation view taken along line V-V of FIG. 3, depicted with the top, inner cuff in the deflated state;

FIG. 6 is another sectional side elevation view taken along line VI-VI of FIG. 4, depicted with the top, inner cuff in the inflated state;

FIG. 7 is a sectional side elevation view of another cervical support and reinforcement device in accordance with the present invention, depicted with a top, inner cuff in a deflated or neutral state;

FIG. 8 is another sectional side elevation view of the cervical support device of FIG. 7, depicted with the top, inner cuff in an inflated or expanded state;

FIG. 9 is a radial, sectional side elevation view of a portion of another expandable or inflatable cuff for a cervical support device;

FIG. 10 is a radial, sectional side elevation view of a portion of another expandable or inflatable cuff for a cervical support device;

FIG. 11 is a radial, sectional side elevation view of a portion of another expandable or inflatable cuff for a cervical support device;

FIG. 12 is a radial, sectional side elevation view of a portion of another expandable or inflatable cuff for a cervical support device, depicted in a deflated or neutral state;

FIG. 13 is another radial, sectional side elevation view of the portion of inflatable cuff of FIG. 12, depicted in an inflated or expanded state;

FIG. 14 is a sectional side elevation view of another cervical support and reinforcement device in accordance with the present invention, depicted with a top, inner cuff in a deflated or neutral state;

FIG. 15 is another sectional side elevation view of the cervical support device of FIG. 14, depicted with the top, inner cuff in an inflated or expanded state;

FIG. 16 is another sectional side elevation view of the cervical support device of FIG. 14, shown in position around a lower portion of a cervix, and depicted with the top, inner cuff in the deflated state;

FIG. 17 is another sectional side elevation view of the cervical support device of FIG. 16, depicting the lower portion of the cervix extending into the opening formed by the top, inner cuff;

FIG. 18 is another sectional side elevation view of the cervical support device of FIG. 17, depicted with the top, inner cuff in the inflated state and engaging or impinging on the exterior of the lower portion of the cervix;

FIG. 19 is a sectional side elevation view of another cervical support and reinforcement device, in accordance with the present invention, the cervical support device depicted with a lower, outer cuff in a deflated or neutral state;

FIG. 20 is another sectional side elevation view of the cervical support device of FIG. 19, depicted with the lower, outer cuff in an inflated or expanded state;

FIG. 21 is a radial, sectional side elevation view of a pair of adjacent expandable or inflatable cuffs, depicted in a deflated or neutral state;

FIG. 22 is another radial, sectional side elevation view of the inflatable cuffs of FIG. 21, depicted with the cuffs in an inflated or expanded state;

FIG. 23 is a top perspective view of another cervical support and reinforcement device in accordance with the present invention, depicted with a top, inner cuff and a lower cuff each in a deflated or neutral state;

FIG. 24 is bottom plan view of the cervical support device of FIG. 23;

FIG. 25 is a top perspective view of another cervical support and reinforcement device, in accordance with the present invention, depicted with a top, inner cuff and a lower cuff each in a deflated or neutral state;

FIG. 26 is an exploded side perspective view of the cervical support device of FIG. 25;

FIG. 27 is an exploded sectional view of the components of the cervical support device of FIG. 25, viewed along line XXVII-XXVII of FIG. 25;

FIG. 28 is another top perspective view of the cervical support device of FIG. 25, depicted with the top, inner cuff in an inflated or expanded state;

FIG. 29 is a sectional side elevation view taken along line XXVI-XXVI of FIG. 25, depicted with the top, inner cuff and the lower cuff each in the deflated state;

FIG. 30 is another sectional side elevation view of the cervical support device of FIG. 29, depicted with the top, inner cuff in the inflated state and the lower cuff in the deflated state;

FIG. 31 is a sectional side elevation view taken along line XXXI-XXXI of FIG. 25, depicted with the top, inner cuff and the lower cuff each in the inflated state;

FIG. 32 is another sectional side elevation view of the cervical support device of FIG. 29 and a quad lumen inflation device coupled with a valve system for inflating the cuffs of the device;

FIG. 32A is an enlarged view of the region designated XXXIIA in FIG. 32;

FIG. 32B is a side perspective view of the quad lumen of FIG. 32;

FIG. 33 is a sagittal view of a patient with a single gestation, including a sectional side view of the cervical support device of FIG. 25 in the vagina of the patient to support the patient's cervix;

FIG. 34 is a radial, sectional side elevation view of an expandable or inflatable cuff of the cervical support device of FIG. 25;

FIG. 35 is a top perspective view of another cervical support and reinforcement device in accordance with the present invention, depicted with a top, inner cuff and a lower cuff each in a deflated or neutral state;

FIG. 36 is a side perspective view of another cervical support and reinforcement device in accordance with the present invention, depicted with a top, inner cuff in a deflated or neutral state;

FIG. 37 is a sectional side perspective view taken along line XXXVII-XXXVII of FIG. 36;

FIGS. 38-42 are radial, sectional side elevation views of exemplary cleats or teeth for expandable or inflatable cuff, in accordance with the present invention;

FIGS. 43-50 are front elevation views of exemplary cleats or teeth for expandable or inflatable cuffs for use with cervical support devices of the present invention;

FIG. 51 is a side perspective view of another cervical support and reinforcement device in accordance with the present invention, depicted with a top, inner cuff and a lower, outer cuff each in a deflated or neutral state;

FIG. 52 is a sectional side perspective view taken along line LII-LII of FIG. 51;

FIG. 53 is another sectional side perspective view of the cervical support device of FIG. 52, depicted with the top, inner cuff in the deflated state and the lower, outer cuff in an inflated or expanded state;

FIG. 54 is another sectional side perspective view of the cervical support device of FIG. 53, depicting a tube and valve assembly for inflating the top and lower cuffs;

FIG. 55 is a bottom side perspective view of another cervical support and reinforcement device in accordance with the present invention, depicted with a top, inner cuff in a deflated or neutral state;

FIG. 56 is a sectional bottom side perspective view of the cervical support device of FIG. 55 taken below and perpendicular to an axial center of the top, inner cuff of the device; and

FIG. 57 is a partial sectional bottom side perspective view taken along line LVII-LVII of FIG. 55, depicted with internal components of the device intact to show internal structure.

### DESCRIPTION OF THE PREFERRED EMBODIMENTS

Referring now to the drawings and the illustrative embodiments depicted therein, various embodiments of a cervical support device are provided for reduction or prevention of preterm labor caused by cervical insufficiency (i.e. shortened or incompetent cervix). The devices are adjustable to apply a compressive force around the exterior of the cervix and may function to alter the angle of the cervix, which may promote an extended gestation of a fetus in patients who experience cervical insufficiency. The devices include one or more expandable (e.g. inflatable) cuffs, which when expanded apply a compressive force to the exterior of the cervix. The cuff may be adjusted by an operator or user (e.g. medical care provider) without removing the device from inside the patient. In some instances where the cervix is partially dilated, opened to the point the cervical mucus plug has fallen out, or in instances of PROM or PPROM, the device can be utilized to compress the cervix to facilitate mucus plug regeneration, which may prevent infection from traveling up the cervix into the uterus. The device may be utilized in a minimally invasive manner to provide support around the lower cervix to support the weight of the uterus and fetus, preferably with little or no stimulation or irritation to the cervix. In other words, the cuff(s) may be left in a neutral or uninflated state. In the event that the patient's cervix continues to shorten and dilate and the minimally invasive approach is not adequate, the cuff(s) may be inflated, or inflated further, to decrease the proximal opening diameter to supply additional support to the external cervix and provide external compressive force around the exterior of the cervix in effort to keep the cervix from opening or dilating further. The term “cuff” and “bladder” are used herein to refer to the expanding annular components of the illustrative embodiments of the present disclosure, including “proximal cuffs”, “distal cuffs”, and “inflatable bladders”, for example. Optionally, the cuffs (either proximal or distal) may be continuous around a circumference of the device, intermittently spaced around a circumference of the device, may expand to define non-circular or non-uniform openings, and/or may be expandable in both lateral (e.g. radial) and oblique (e.g. non-radial) directions. It will be appreciated that the cervical support device may be utilized to support the cervix, uterus, and fetus, even with the cuff(s) in a neutral or uninflated state.

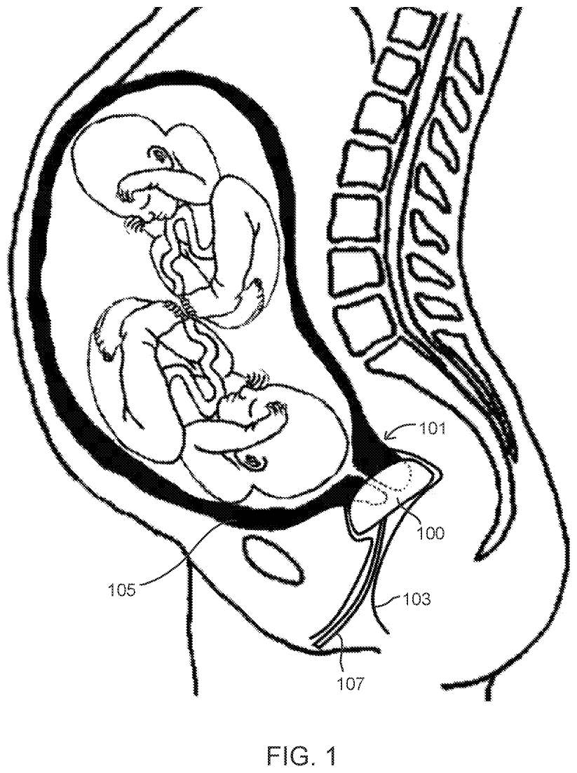

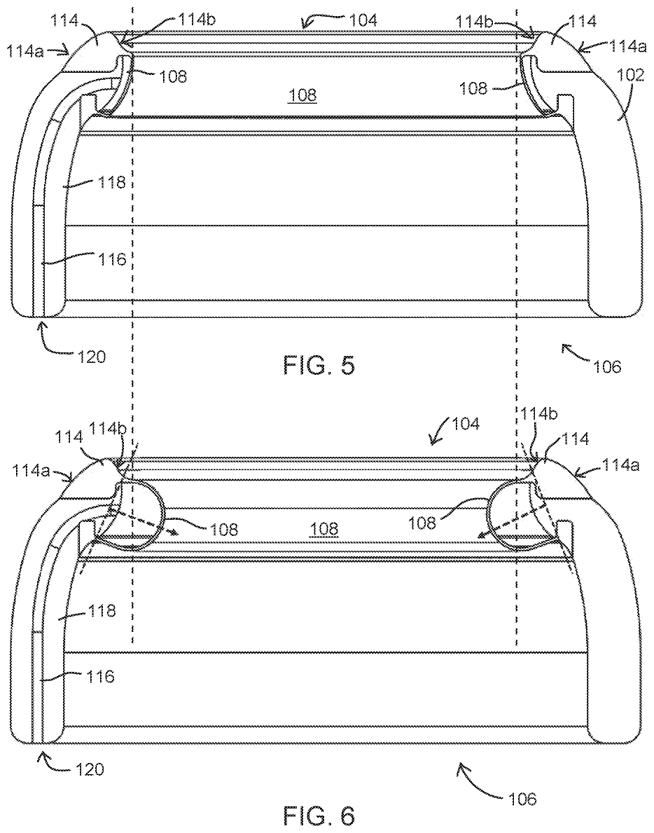

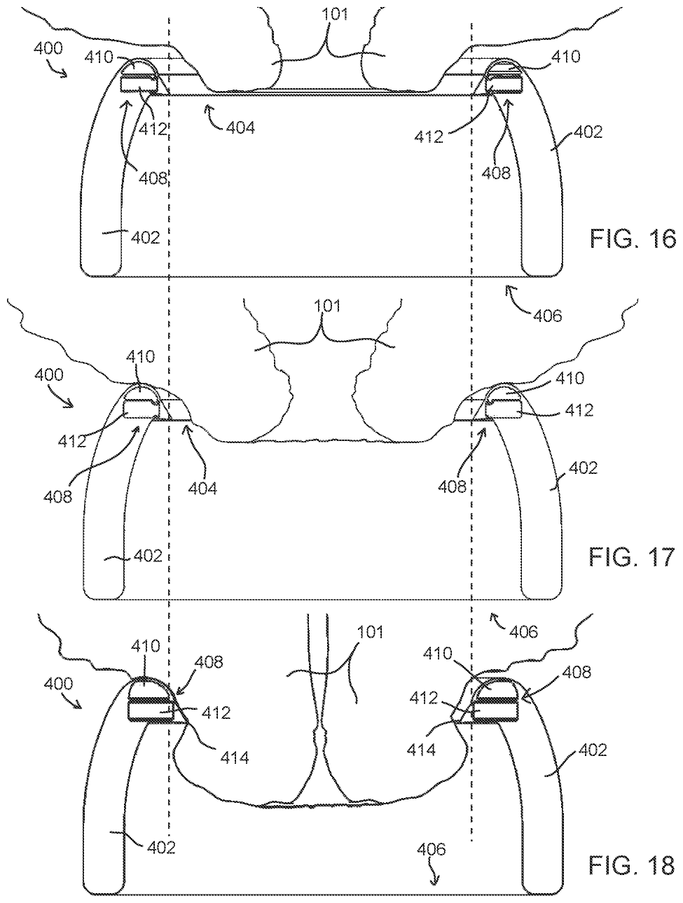

Referring to the illustrative embodiments of FIGS. 1-6, a cervical support and reinforcement device 100 is provided for the reduction or prevention of preterm labor. The device 100 includes a flexible, semi-spheroid or semi-sphere-like body 102 having an interior passageway with an upper or proximal opening 104 at an upper portion of the body 102 and a lower or distal opening 106 at a lower portion of the body 102 (FIGS. 5 and 6). In the illustrative embodiment, the proximal opening 104 is smaller in diameter than the distal opening 106 and is dimensioned to receive and encircle the lower, exterior portion of the patient's cervix 101 when the device is placed in the patient's vagina 103 proximate the base of the cervix (FIGS. 1 and 2). For reference purposes, FIGS. 16-18 provide a sequential illustration of the interaction between a cervical support device 400 (which is similar to cervical support device 100 of FIGS. 1-6) and the patient's cervix 101 as the device is placed and engages with the cervix. The exterior perimeter of the lower portion of the body 102 is dimensioned to engage or interact with the patient's interior vaginal wall (see FIGS. 1 and 2). In other words, the exterior perimeter of the body 102 at the distal opening 106 engages the vaginal wall to secure or support the device 100 in sufficient proximity to the cervix 101.

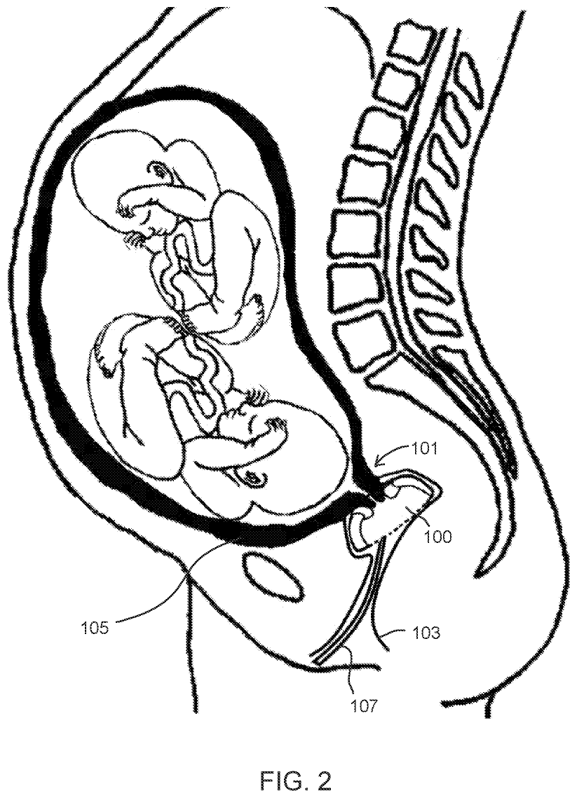

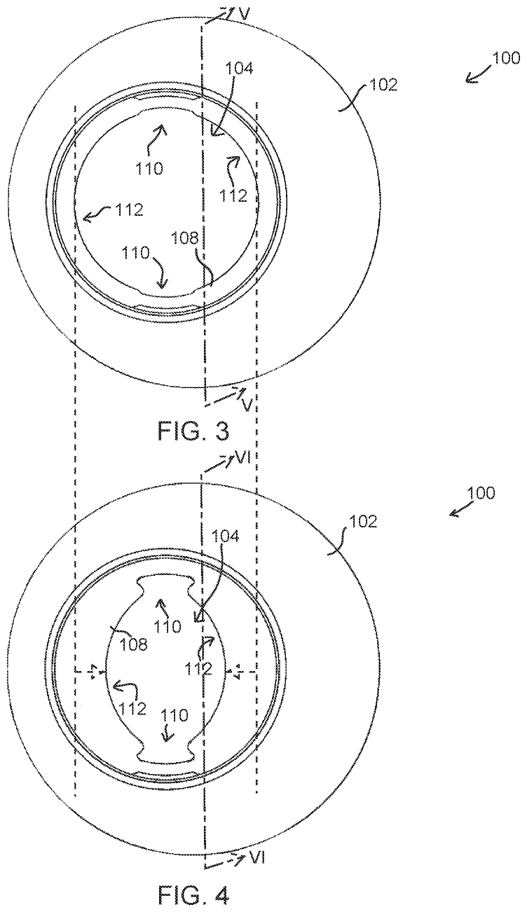

An upper or proximal cuff or bladder 108 is provided around the circumference of the proximal opening 104. The upper cuff 108 is expandable, e.g. inflatable with a fluid such as saline or air, to constrict or circumscribe the exterior of the lower portion of the cervix 101. The proximal cuff 108 is shown in a normal, neutral, or deflated state in FIGS. 3 and 5, and shown in an expanded or inflated state in FIGS. 4 and 6, in which portions of the cuff 108 have been expanded toward a center of the proximal opening 104. In the inflated state, the proximal cuff 108 encroaches or impinges on the exterior of the cervix 101 and thereby applies an external force or pressure on the cervix which may assist in retaining the cervical opening or OS in a sufficiently closed condition, permitting the fetus to remain in utero for a longer duration. The area of the proximal opening 104 is significantly reduced when the proximal cuff 108 is inflated. A pair of phantom lines are provided between sequential FIGS. 3 and 4 and sequential FIGS. 5 and 6 to highlight the relative expansion of the proximal cuff 108 from its neutral state in FIGS. 3 and 5 to its expanded state in FIGS. 4 and 6. In the illustrative embodiment, the proximal cuff 108 is coupled to the proximal end of the wall of body 102, such as in the form of a tongue-and-grove or dovetail fit arrangement. However, it will be appreciated that the cuff 108 may integrally formed with the wall of body 102, such as via a unitary molding process or an over-molding process, for example.

As best illustrated in FIGS. 3 and 4, proximal cuff 108 includes two recessed portions 110 at opposing sides of the proximal opening 104 and two enlarged portions 112 at other opposing sides of the proximal opening 104 and formed between the recessed portions 110. In the neutral state, the proximal cuff 108 defines a generally circular shape (FIG. 1). As the proximal cuff 108 expands, the enlarged portions 112 expand toward the axial center of the proximal opening 104 and the recessed portions 110 remain substantially static, unchanged, or unexpanded. As such, in the expanded state, the recesses 110 and enlarged portions 112 cooperate to form a non-circular inner perimeter of the proximal cuff 108 (FIG. 5). With the proximal cuff 108 in the expanded state, the area of the proximal opening 104 is decreased as compared to its area with the proximal cuff 108 in the neutral state. The recessed portions 110 are positioned to receive portions of the cervix 101 containing the major blood vessels that supply blood to the end of the cervix, as such the recessed portions 110 enable blood to flow into the lower tissue of the cervix extending through the proximal opening 104 while the proximal cuff 108 is expanded and providing compressive force to the cervix.

As sequentially illustrated in FIGS. 5 and 6, as the proximal cuff 108 expands, it does so both radially inward toward the center of the proximal opening 104 as well as downward in relation to the transverse plane of the proximal opening 104. In other words, with the device 100 placed in the vagina proximate the cervix 101, as the proximal cuff 108 expands it constricts the exterior of the cervix while applying a downward force (i.e. away from the uterus 105) on the cervix. The downward angle of expansion of the proximal cuff 108 may act to pull or push the cervix 101 downward, away from the uterus 105, to mitigate or prevent the cervix from slipping out of the cuff either due to being displaced by the cuff's increasing volume during expansion or due to the cervix continuing to shorten over time. In other words, the combination of transverse and downward constricting forces may more effectively engage and constrain the exterior of the cervix 101, as compared to if the constricting force was applied only in the transverse direction. The angle of expansion of the proximal cuff 108 away from the transverse plane of proximal opening 104 may be selected from a range of between about one-half degree (0.5°) and eighty-nine and a half degrees (89.5°).

It will be appreciated that the entirety of the proximal cuff 108 may be formed of the same material (e.g. flexible silicone), the different portions 110 and 112 of the cuff 108 may be formed of different materials (e.g. the enlarged portions 112 of flexible silicone and the recessed portions 110 of medical grade plastic), and/or the different portions 110 and 112 of the cuff 108 may be formed of differing compositions of a material (e.g. the enlarged portions 112 of flexible silicone and the recessed portions 110 of semi-rigid silicone having a higher durometer than the silicone of the enlarged portions 112).

The device 100 includes a raised, annular rim, ridge, or lip 114 at the upper portion of the body 102, which rim 114 extends upward above or proud of the proximal cuff 108 (i.e. toward the uterus 105 when the device is placed in the patient's vagina; see FIGS. 5 and 6). The annular rim 114 defines the uppermost or most proximal edge of the device 100 and includes a sloped exterior wall 114a extending from the top of the rim 114 toward the exterior of the body 102, and a sloped interior wall 114b extending from the top of the rim 114 toward the interior perimeter of the proximal cuff 108 (in the cuff's neutral state; see FIGS. 5 and 6)). The sloped interior wall 114b encourages entry of the cervix 101 into the proximal opening 104 by funneling or guiding the end of the cervix along the sloped interior wall during placement of the device in the vagina. The sloped interior wall 114b may be angled as much as about eighty degrees (80°) in relation to the longitudinal axis of the device 100 while providing some guidance to the cervix 101. Preferably, the angle of the sloped interior wall 114b is between about five degrees (5°) and about sixty degrees) (60°. In some embodiments, the raised lip may extend as much as about ten millimeters (10 mm) above the upper or proximal edge of the proximal cuff 108. Preferably, the rim 114 is between about one millimeter (1 mm) and about four millimeters (4 mm) above the upper or proximal edge of the proximal cuff 108.

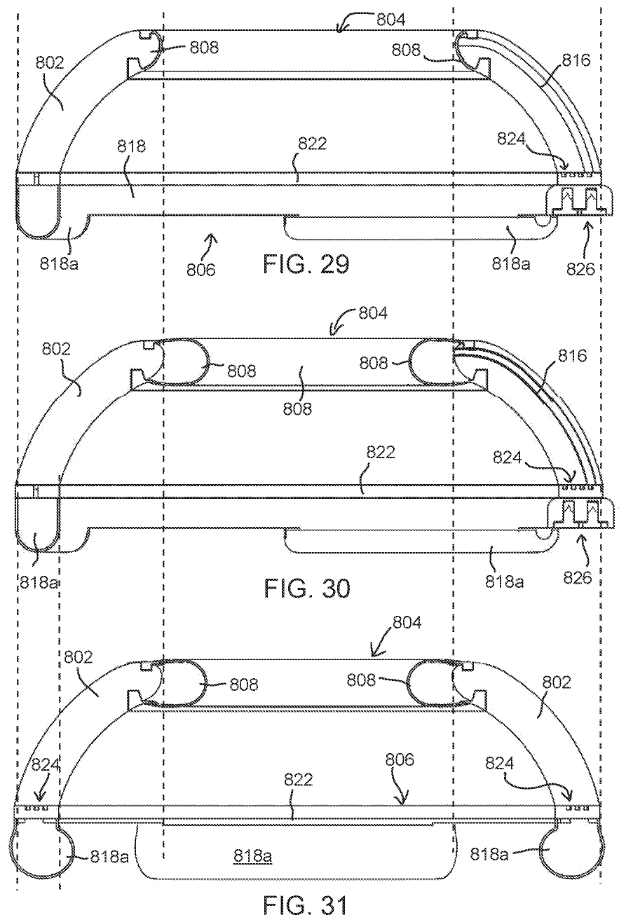

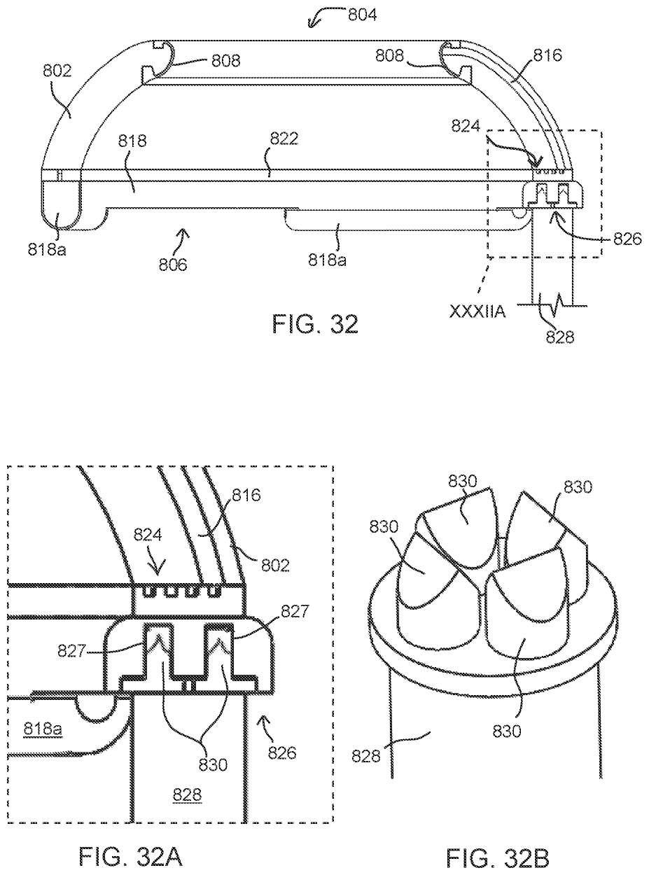

A fluid conduit 116 is provided through a wall 118 of the body 102, such as shown in FIGS. 5 and 6. The conduit 116 is in fluid communication with the proximal cuff 108 to permit a care provider to inflate or deflate the cuff 108 as needed to provide adequate support to the cervix 101. An inlet 120 is provided at an exterior portion of the body 102 where an inflation/deflation device may be removably coupled to the device 100 in order to inflate or deflate the cuff 108 without removing the device 100 from the patient's vagina. Optionally, a valve may be provided at or near the inlet 120 to regulate the flow of fluid into and out of the conduit 116. An exemplary valve system 826 is illustrated in FIGS. 29 and 30, and an inflation/deflation device 827 (including a tube 828 and valves 830) is illustrated in FIG. 32, which are described in further detail below.

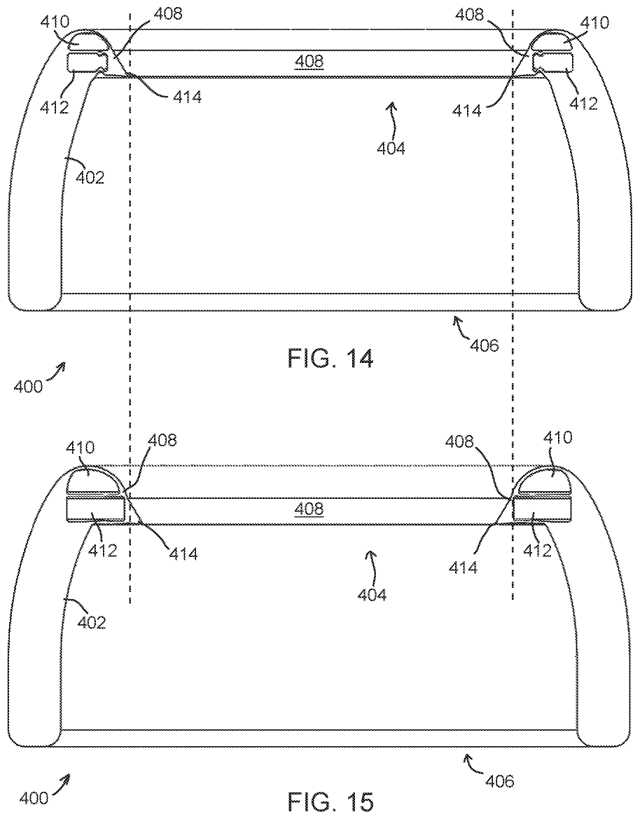

It will be appreciated that while in some of the illustrated embodiments the proximal cuff expands downward or away from the transverse plane of the proximal opening, other embodiments are contemplated in which the proximal cuff expands substantially along the transverse plane, such as depicted in the illustrative embodiments of similar cervical support devices 400 and 800 shown in FIGS. 14-18 and 29-30, respectively, and described in further detail below. For example, a proximal cuff 808 may be configured to expand in a purely radial and concentric manner as sequentially depicted for device 800 in FIGS. 29 and 30.

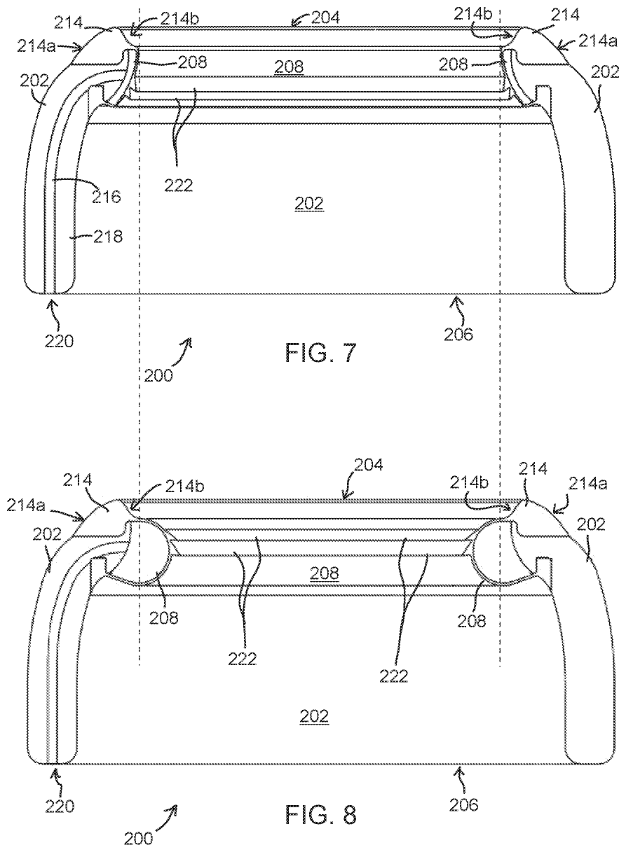

Referring now to the illustrative embodiment of FIGS. 7 and 8, another cervical support and reinforcement device 200 is provided for the reduction or prevention of preterm labor. The device 200 is similar to device 100 described above, but includes some alternative and/or additional components and functions. Similar to the components of device 100, device 200 includes a main body 202, a proximal opening 204, a distal opening 206, an expandable annular cuff 208, a raised, annular rim 214, and a fluid conduit 216 in a body wall 220. As compared to device 100, cervical support device 200 includes mechanical anchor elements, in the form of a pair of annular ridges, cleats, or teeth 222, along the interior perimeter of the proximal cuff 208. Due to the use environment of the device 200 within the vaginal canal and around the outer cervix, which each have a relatively low coefficient of friction due to the bodily fluids present (e.g. discharge and mucus), mechanical anchor elements may discourage the device 200 from slipping off the cervix 101. Mechanical anchor elements may also be provided to engage the vaginal wall 103. Accordingly, the ridges 222 are configured to provide mechanical anchor elements for gripping the exterior of the cervix 101 (see placement example in FIGS. 1 and 2) to provide additional grip to retain the cervix in an elongated and closed state and mitigate or prevent the cervix from slipping out of the proximal cuff 208. It is contemplated that multiple rows of cleats or ridges may be provided such that as the cuff expands, the compressive force directed onto the cervix is substantially focused on the edge of one or more of the ridges to ensure sufficient interaction between the cervix and cuff. The additional rows of ridges may aid in keeping the cervix from slipping out of the cuff as the cuff expands, which may otherwise occur due to the low coefficient of friction of the surface of the cervix.

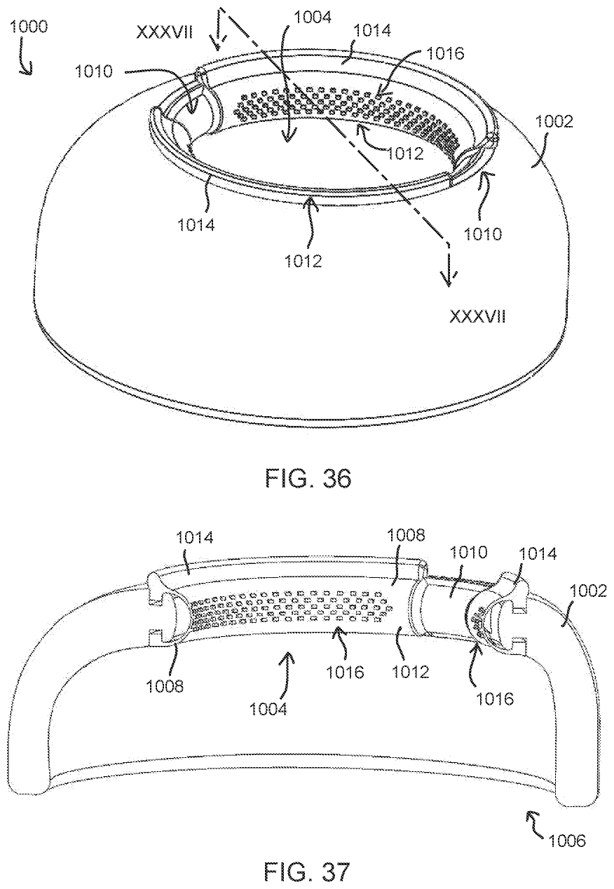

The ridges 222 may be continuous or uninterrupted around the entire interior perimeter of the proximal cuff 208. However, it will be appreciated that the ridges 222 may be segmented and spaced along the interior perimeter of the proximal cuff (see below for examples of segmented cleats for devices 1000 and 1100 of FIGS. 36-37 and 51-54). In an embodiment having segmented ridges, the ends of each ridge segment may be chamfered for comfort and/or to concentrate more of the compressive force from the cuff to a smaller area of the cervix 101. The ridge height or depth may be between about one-tenth millimeter (0.1 mm) and ten millimeters (10 mm). Preferably, each of the ridges 222 may be formed of a similar material as the proximal cuff 208 (e.g. flexible silicone) and integrally formed or molded with the cuff 208. However, it is contemplated that the ridges may be formed of different material than the cuff, such as a rigid polymer (e.g. polyether ether ketone (PEEK), a higher durometer silicone than that used for the cuff, or a semi-flexible metal (e.g. 316L stainless steel), for example. While it may be preferable for manufacturing and durability purposes to form the ridge along with the cuff, the ridges may be formed separate from the cuff and then adhered to the cuff, such as through an over-molding process, bonding, or fixation with a mechanical fastener.

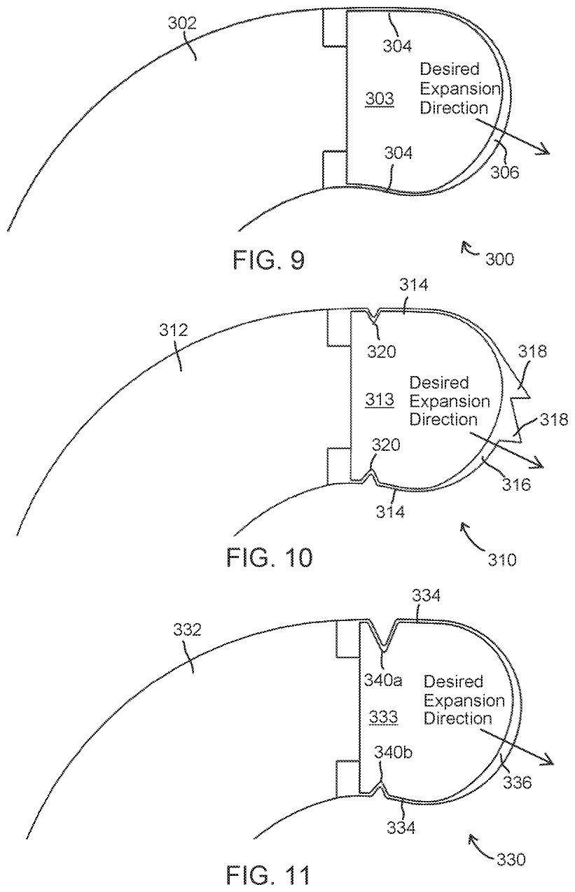

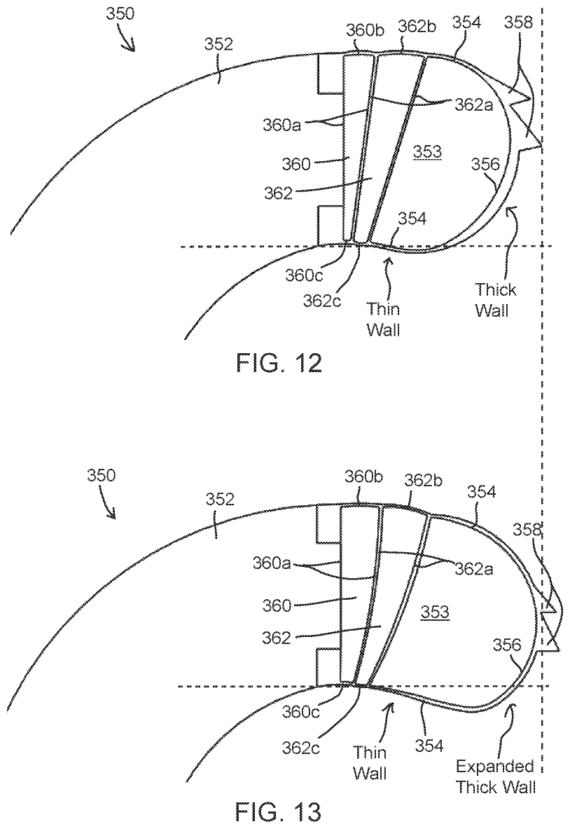

In the various cervical support devices of the present disclosure, the proximal cuffs may utilize various structural aspects to achieve the downward expansion of the cuff relative to the transverse plane of the proximal opening, as will be discussed in further detail below. For example, the cuff may be formed with a non-uniform wall thickness along a radial cross-section thereof. Upon expansion, the cuff with non-uniform wall thickness expands in the direction of the thickest portion of the wall (see exemplary cuffs depicted in FIGS. 9-13 discussed in further detail below, and in particular sequential FIGS. 12 and 13 depicting expansion of the cuff at the thickest portion of the cuff wall). In embodiments having a cuff forming a non-circular proximal opening, the enlarged portions may utilize a non-uniform wall thickness, while the recessed portions may have generally uniform wall thickness, for example.

A section view of an exemplary expandable cuff 300 is illustrated in FIG. 9, and is shown coupled to a proximal portion of a wall 302 of a cervical support device (such as devices 100 or 200 described above). The cuff 300 includes a main inflatable bladder or chamber 303 that is defined by upper and lower thin-walled portions 304 and a thicker wall or thick-walled portion 306 between the thin-walled portions 304 at a proximal portion of the cuff 300. The upper and lower thin-walled portions 304 are generally parallel with one another and the transverse plane of the proximal opening formed by the cuff. In this configuration, due to the properties of the flexible silicone, upon inflation of the cuff the thick-walled portion 306 will expand more than the thin-walled portions 304 such that the cuff 300 effectively expands both radially toward the center of the proximal opening and downward away from the transverse plane of the proximal opening. During inflation, the thin-walled portions 304 will expand more quickly initially than the thick-walled portion 306, until the thin-walled portions 304 each reach maximum expansion, at which point they will no longer expand, and the remaining expansion of the cuff 350 will occur at the thick-walled portion 306. Exemplary wall thicknesses for the cuff include the thin-walled portions 304 ranging between about 0.004 inches and 0.020 inches thick, while the thick-walled region 306 ranges between about 0.010 inches to 0.100 inches thick. It will be appreciated that the different portions, i.e. thin-walled portions 304 and thick-walled portion 306, may be formed of silicones having different durometers, which may further facilitate expansion of the cuff 300 in the desired direction.

Another exemplary expandable cuff 310 is illustrated in FIG. 10, and is shown coupled to a proximal portion of a wall 312 of a cervical support device (such as devices 100 or 200 described above). The cuff 310 includes similar or substantially identical structure to exemplary cuff 300, including a main inflatable bladder or chamber 313, thin-walled portions 314 and a thick-walled portion 316, which function in substantially the same manner as the corresponding aspects of cuff 300. In comparison to cuff 300 of FIG. 9, the cuff 310 illustrated in FIG. 10 includes a pair of ridges, cleats, or teeth 318, which are similar in structure and function to the ridges 222 described above for cervical support device 200 and illustrated in FIGS. 7 and 8. The cuff 310 also includes a pair of buckled wall segments 320 in respective thin-walled portions 314 (FIG. 10). In the neutral, unexpanded state, the buckled wall segments 320 of the cuff 310 retain their buckled shape, as illustrated in FIG. 10. As the cuff 310 is inflated, the buckled wall segments 320 extend and flatten to provide the initial expansion of the cuff 310 and permitting the cuff to expand radially toward the center of the proximal opening. Once the buckled wall segments 320 have substantially flattened out, the remainder of the expansion of the cuff 310 may occur at the thick-walled portion 316. It will be appreciated that multiple buckled wall segments may be provided at the upper and/or lower portions of the cuff to permit the cuff to expand in a desired direction and/or through a desired displacement. Multiple buckled wall segments may permit extended expansion of the cuff 310 without the cuff reaching the limit of elastic deformation. It will also be appreciated that a buckled wall segment may be provided at only the upper or lower portion of the cuff, to permit the cuff to expand in a desired direction and/or through a desired displacement.

Another exemplary expandable cuff 330 is illustrated in FIG. 11, and is shown coupled to a proximal portion of a wall 332 of a cervical support device (such as devices 100 or 200 described above). The cuff 330 includes similar or substantially identical structure to exemplary cuffs 300 and 310, including a main inflatable bladder or chamber 333, thin-walled portions 334, and thick-walled portions 336 which function in substantially the same manner as the corresponding aspects of cuffs 300 and 310. In comparison to cuff 300 of FIG. 9, the cuff 330 includes a pair of buckled wall segments 340a and 340b in respective thin-walled portions 334 (FIG. 11). In comparison to cuff 310 of FIG. 10, the cuff 330 illustrated in FIG. 11 does not include ridges, cleats, or teeth. Also, cuff 330 includes a larger buckled wall segment 340a at the upper thin-walled portion 334 and a smaller buckled wall segment 340b at the lower thin-walled portion 334. In the neutral, unexpanded state, the buckled wall segments 340a and 340b of the cuff 330 retain their buckled shape, as illustrated in FIG. 11. As the cuff 330 is inflated, the buckled wall segments 340a and 340b extend and flatten. Because segment 340a is larger than segment 340b, it is extendable to a longer length than segment 340b, thus permitting the cuff 330 to expand both radially toward the center of the proximal opening and downward away from the transverse plane of the proximal opening.

Yet another exemplary expandable cuff 350 is illustrated in FIGS. 12 and 13, and is shown coupled to a proximal portion of a wall 352 of a cervical support device (such as devices 100 or 200 described above). The cuff 350 includes similar or substantially identical structure to exemplary cuffs 300 and 310, including a main inflatable bladder or chamber 353, thin-walled portions 354, thick-walled portions 356, and a pair of ridges, cleats, or teeth 358, which each function in substantially the same manner as the corresponding aspects of cuffs 300 and 310. In comparison to cuff 310 of FIG. 10, the cuff 350 illustrated in FIGS. 12 and 13 does not include buckled wall segments. In comparison to cuff 300 of FIG. 9, the cuff 350 includes a pair of inflatable bladders or chambers 360 and 362, in addition to the main inflatable chamber 353 (FIGS. 12 and 13). The pair of chambers 360 and 362 are adjacent to and substantially co-axial with one another and the main chamber 353. In the illustrative embodiment, the chambers 360 and 362 are defined as elongated trapezoids with elongated side walls 360a and 362a, and upper walls 360b and 362b that are longer than bottom walls 360c and 362c. In the neutral, unexpanded state, the longer top walls 360b and 362b, position the cuff 350 in an initially downward facing angle to facilitate the downward expansion on the cuff relative to the transverse plane of the proximal opening. As the cuff 350 is inflated, the pair of chambers 360 and 362 may extend somewhat toward the radial center of the proximal opening, or may remain substantially unchanged or uninflated. During inflation, the main chamber 353 extends both radially inward toward the axial center of the proximal opening and downward relative to the transverse plane of the proximal opening. The relative radial and downward movements of the inflated cuff 350 can be see with reference to phantom lines provided in and between sequential FIGS. 12 and 13. The chambers 353, 360, and 362 may be in fluid communication with one another such that any inflation of the cuff 350 inflates all three chambers together, or each chamber may be individually sealed and in fluid connection with a dedicated fluid conduit such that the chambers are individually addressable (e.g. inflatable or deflatable) by a provider.

A similar cuff is contemplated that is similar to that of the expandable cuff 350 described above and illustrated in FIGS. 12 and 13, however instead of the long walls of the additional chambers being located adjacent one another at the respective upper walls 360b and 362b as shown in FIGS. 12 and 13, the longer walls would be offset such that one chamber would have a long upper wall 360b and a short lower wall 360c similar to that shown in FIGS. 12 and 13, and the adjacent chamber would have a short upper wall and a long lower wall. In such an embodiment, as the chambers of the cuff are inflated, the two adjacent upper and lower walls of the additional chambers may extend in a manner similar to a sinusoidal wave. This allows a care provider to make minor adjustments to the angle of the expanded cuff to adjust and fine tune the direction of the compressive force applied around the cervix, or may permit the provider to reposition the cervix within the cuff. In yet another contemplated embodiment, one of the additional chambers may have equal upper and lower wall lengths, while the second additional chamber has non-uniform upper and lower wall lengths (e.g. the trapezoidal chambers 360 and 362 as illustrated in FIGS. 12 and 13), such that the first chamber expands substantially radially toward the axial center of the proximal opening, and the second cuff expands both radially inward toward the axial center of the proximal opening and downward relative to the transverse plane of the proximal opening.

Referring now to FIGS. 14-18, another cervical support and reinforcement device 400 is provided for the reduction or prevention of preterm labor. The device 400 is similar to devices 100 and 200 described above, but includes some alternative and/or additional components and functions. Similar to the components of devices 100 and 200, device 400 includes a main body 402, a proximal opening 404, a distal opening, 406, and an expandable annular cuff 408 (see placement example in FIGS. 1 and 2). The proximal cuff 408 is similar to cuff 350 described above and illustrated in FIGS. 12 and 13 in that cuff 408 includes multiple inflatable bladders or chambers 410 and 412, however, instead of being positioned side-by-side like bladders 360 and 362, an upper first bladder 410 is positioned above a lower second bladder 412. The bladders 410 and 412 are integrally formed (e.g. over-molded) into an upper or proximal portion of the wall 402 of device 400, or unitarily formed with the upper or proximal portion of the wall 402. FIG. 14 shows the device 400 with both cuff bladders 410 and 412 in a neutral or deflated state, and FIG. 15 shows the device 400 with both cuff bladders 410 and 412 in an inflated or expanded state.

The bladders 410 and 412 may be individually addressable and are configured such that if the upper bladder 410 is inflated independently, the cuff 408 expands in a generally upward manner (i.e. toward the uterus 105), and if the lower bladder 412 is inflated independently, the cuff expands generally radially inward, toward the axial center of the proximal opening 404. As such, the independent bladders 410 and 412 of cuff 408 may permit a care provider to fine tune or adjust positional aspects of the device 400 without the need to manually adjust the device's position intravaginally and risk potential further irritation to the cervix 101 or vagina 103. For example, with the device 400 placed in the vagina 103 and with the lower portion of the device engaged with the ligaments and muscles of the vaginal wall (see placement example in FIGS. 1 and 2), the upper chamber 410 may be initially inflated or expanded to apply a force to the cervix 101 that is normal to the transverse plane of the proximal opening 404, or in other words to apply a force to the cervix 101 that is directed substantially upward toward the uterus 105, which may act to displace more of the cervical tissue and thereby urge it into the proximal opening 404. With the cervix 101 sufficiently embedded in the proximal opening 404, a provider may then inflate the second, lower chamber 412, to apply a constrictive, compressive force around the cervix. An annular ridge, tooth, or cleat 414 (FIGS. 14 and 15) is provided at the interior perimeter of proximal cuff 408 such that as the lower bladder 412 expands, the ridge 414 encroaches onto and engages the exterior of the cervix 101 (see FIG. 18). It is contemplated that the perimeter walls of the bladders 410 and 412 may include expansion elements formed therein to provide or facilitate the desired direction of expansion for each respective bladder. For example, the perimeter of the bladders 410 and 412 may include expansion elements in the form of one or more buckled or folded walls or wall segments (such as similar to buckled wall portions 320 and 340a and 340b, respectively illustrated in FIGS. 10 and 11), which upon expansion of the respective bladders, bias the expansion of the respective bladders towards a desired direction (e.g. upward toward the uterus 105 or radially toward an axial center of the proximal opening 404). As another example, particular portions of the bladder walls may include perforations, which upon expansion of the respective bladders, bias the expansion of the respective bladders towards a desired direction.

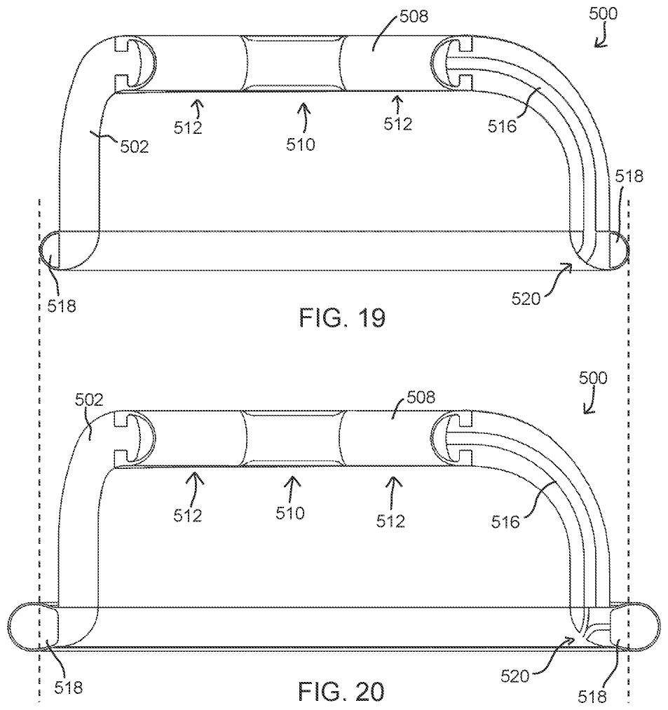

Referring now to FIGS. 19 and 20, another cervical support and reinforcement device 500 is provided for the reduction or prevention of preterm labor. The device 500 is similar to devices 100, 200, and 400 described above, but includes some alternative and/or additional components and functions, with the more significant differences described below. Similar to the components of devices 100, 200, and 400, device 500 includes a main body 502, a proximal opening 504, a distal opening 506, an expandable annular cuff 508 proximate the proximal opening, recessed portions of the proximal cuff 508, enlarged portions of the proximal cuff 510, and a fluid conduit 516 (see placement example in FIGS. 1 and 2).

Another or second expandable, annular, cuff 518 is provided proximate the distal opening 506 and/or exterior wall of device 500 (FIGS. 19 and 20). Cuff 518 is hereinafter referred to as the distal cuff 518 and is provided for expanding into and engaging the wall of the vagina 103 to provide additional grip or support to retain the device 500 in the desired position adjacent the cervix 101. It will be appreciated that the distal cuff 518 may include similar structures and functions to cuffs 108, 208, 300, 310, 330, 350, and/or 408, described above, such as buckled wall portions 320, 304a, or 340b (FIGS. 10 and 11), non-uniform wall thickness as illustrated with expandable cuffs 300, 310, 330, and 350 (FIGS. 9-13), and/or two or more bladders as illustrated with expandable cuff 350 (FIGS. 12 and 13), and expandable cuff 408 (FIGS. 14-18), for example. FIG. 19 depicts the device 500 with the proximal cuff 508 and the distal cuff 518 each in a neutral or uninflated state, and FIG. 20 depicts the device 500 with the proximal cuff 508 in a neutral or uninflated state and the distal cuff 518 in an inflated or expanded state. During inflation, the distal cuff 518 expands outward, away from the axial center of the distal opening 506, and therefore toward the vaginal wall 103. The outward expansion of distal cuff 518 is illustrated in sequential FIGS. 19 and 20, with phantom lines added to more clearly show the relative movement of the cuff 518 in an expanded state depicted in FIG. 20 relative to its neutral state depicted in FIG. 19.

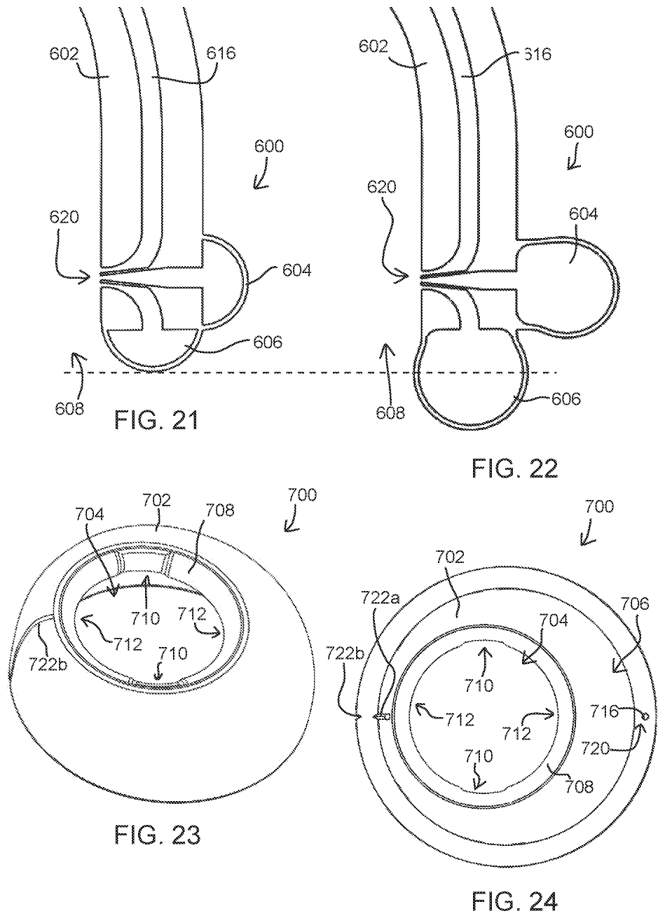

An exemplary expandable cuff system or device 600 is illustrated in FIGS. 21 and 22 and is coupled to a distal portion and/or exterior of a wall 602 of a cervical support and reinforcement device (such as devices 100, 200, 400, or 500 described above). The cuff system 600 includes a pair of inflatable, annular bladders or chambers, including a first or radial bladder 604 and a second or transverse bladder 606. The radial bladder 604 is positioned around an exterior perimeter of the distal portion of the wall 602, and the transverse bladder is positioned along a distal end of the wall 602. Upon inflation, the radial bladder 604 expands outward, away from the axial center of the distal opening 608 of the device in order to provide additional grip or support along the vaginal wall. Upon inflation, the transverse bladder 606 expands downwardly away from the distal end of the wall 602. In other words, the transverse bladder 606 expands in the direction opposite the uterus. A phantom line is provided between sequential FIGS. 21 and 22 to more clearly show the relative expansion of the transverse bladder 606 from its neutral state in FIG. 21 to its expanded state in FIG. 22. In its expanded state, the transverse bladder 606 provides an increase in the overall height of the device 600 which may permit a provider to further engage the cervix 101 a proximal cuff at an upper or proximal end of wall 602 and/or provide additional support to the cervix 101 and bottom of the uterus 105.

It will be appreciated that either or both of the radial bladder 604 or the transverse bladder 606 may include similar structures and functions to cuffs 108, 208, 300, 310, 330, 350, 408, 508 and/or 518, described above, such as buckled wall portions 320, 304a, or 340b (FIGS. 10 and 11), non-uniform wall thickness as illustrated with expandable cuffs 300, 310, 330, and 350 (FIGS. 9-13), and/or two or more smaller bladders as illustrated with expandable cuffs 350 and 408 (FIGS. 12-13 and 14-18, respectively), for example. While the cuff system 600 is illustrated as having two bladders 604 and 606, it will be appreciated that a single bladder or chamber may provide sufficient vaginal wall engagement while also increasing the overall height of the device 600. For example, expansion elements may be provided along particular walls of the single bladder to provide the desired directional expansion. It is contemplated that the device 600 (as well as any of the previous or following embodiments of a cervical support device) may be placed in the patient in an inverted manner in which the distal end faces and engages with the cervix 101 to support the cervix and the uterus 105. In an inverted orientation, the distal cuff of the device is positionable in contact with the upper vaginal walls and the fornix region (i.e. the upper portions of the vagina, which extend into recesses created between the vaginal wall and the exterior of the lower portion of cervix) such that as the distal cuff expands, it may provide support to the uterus 105 at the location where a prolapse would typically occur. In an embodiment where the distal cuff expands in the transverse direction, the distal cuff would distribute the load of the uterus 105 to a larger surface area along the vaginal wall (relative to the device oriented in a standard orientation with the proximal cuff toward the uterus), which may reduce the risk of medical complications, such as from pressure sores and/or necrosis. In an embodiment where the distal cuff expands both in the transverse direction and away from the main device body (e.g. toward the uterus 105), the distal cuff may provide upward support (which may be customized as needed) to the uterus while distributing the load of the uterus 105 to a larger surface area.

FIGS. 23 and 24 illustrate another exemplary cervical support and reinforcement device 700 that is similar or substantially identical to cervical support device 100 described above and illustrated in FIGS. 1-6. Device 700 includes interior and exterior relief notches 722a and 722b formed into interior and exterior portions of the wall 718. The notches 722a and 722b extend substantially between the distal end of the wall and the proximal cuff 708. Notches 722a and 722b provide increased flexibility of the wall 718, which may facilitate collapsing or folding the device 700 to facilitate insertion of the device into the patient's vagina. For example, a provider may be able to fold the device such that it forms a leading edge or point with reduced cross sectional area to more easily project through the vaginal opening. In addition to aiding in insertion, the notches 722a and 722b may also provide a visual indication of which side is intended for initial insertion into the patient.

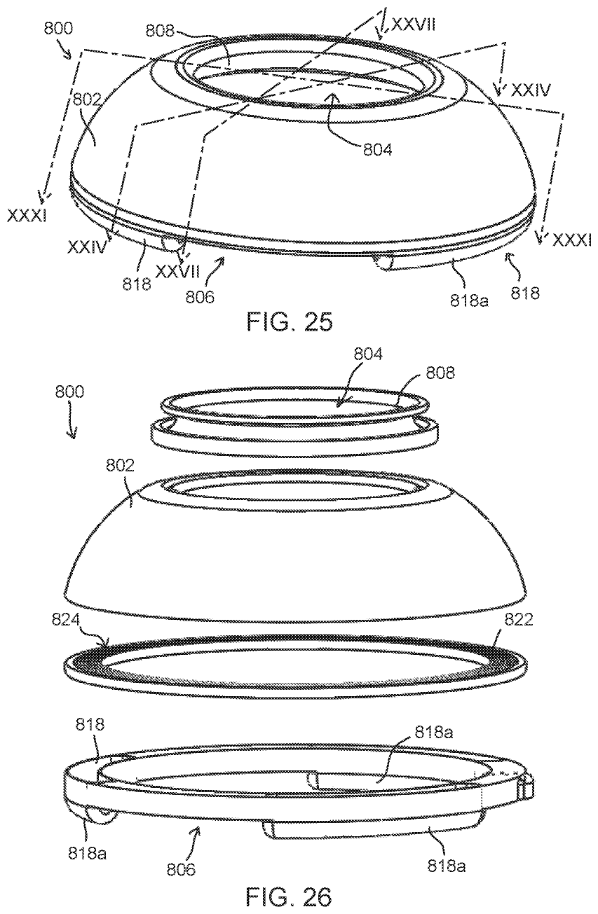

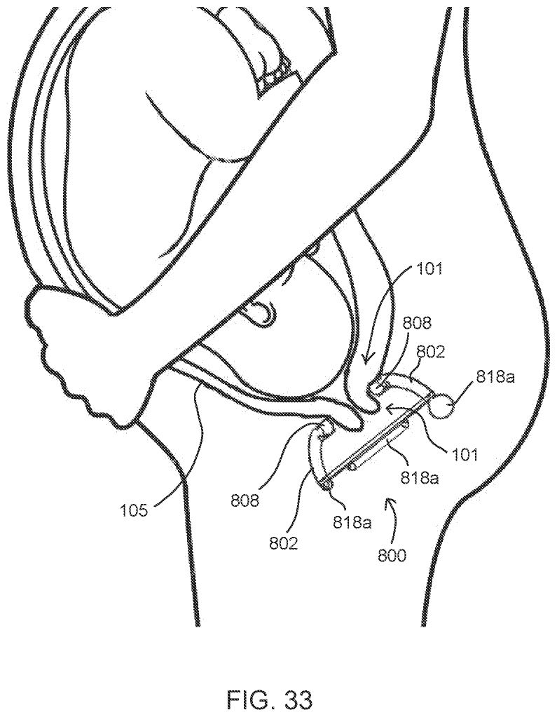

Referring now to FIGS. 25-33, another cervical support and reinforcement device 800 is provided for the reduction or prevention of preterm labor. The device 800 is similar to devices 100, 200, 400, 500, and 700 described above, but includes some alternative and/or additional components and functions, with the more significant differences described below. Similar to the components of devices 100, 200, 400, 500, and 700, device 800 includes a main body 802, a proximal opening 804, a distal opening 806, an expandable annular cuff 808 proximate the proximal opening 804, and a fluid conduit 816 (FIGS. 29 and 30), for example (see placement example in FIGS. 1 and 2). Similar to the components of device 500, and as best illustrated in FIG. 28, the proximal cuff 808 of device 800 includes recessed portions 810 and enlarged portions 812. However, instead of two respective sets of recessed and enlarged portions, proximal cuff 808 includes a set of three recessed portions 810 and a set of three enlarged portions 812, which are uniformly distributed around the proximal portion of the body 802 and define the proximal opening 804. Also similar to the components of device 500, device 800 includes an expandable annular cuff or cuff system 818 proximate the distal opening 806, in which the distal cuff 818 is operable to expand downward, away from the distal end of the wall, and away from the uterus 105. The distal cuff 818 includes three segments of inflatable bladders 818a that are evenly spaced around the circumference of the distal end of the body 802 (FIGS. 25-31). However, it will be appreciated that distal cuff 818 may be provided with more or fewer bladder segments, or the distal cuff 818 may be formed of a single, continuous, uninterrupted bladder. FIG. 29 depicts the device 800 with the proximal cuff 808 and the distal cuff 818 each in a neutral or uninflated state, FIG. 30 depicts the device 800 with the proximal cuff 808 in an inflated or expanded state and the distal cuff 818 in a neutral or uninflated state, and FIG. 31 depicts the device 800 with the proximal cuff 808 and the distal cuff 818 each in an inflated or expanded state. An example of selective inflation of the bladders 818a is depicted in FIG. 33, in which one of the bladders 818a of the distal cuff 818 is inflated more than the other two bladders 818a.

During inflation, the proximal cuff 808 expands substantially inward, toward the axial center of the proximal opening 804. The inward expansion of proximal cuff 808 is illustrated in sequential FIGS. 29 and 30, with phantom lines added to more clearly show the relative movement of the cuff 808 in an expanded state depicted in FIG. 30 relative to its neutral state depicted in FIG. 29. During inflation, the distal cuff 818 expands partially inward toward the axial center of the distal opening 806 and partially outward away from the axial center of the distal opening 806, and therefore toward the vaginal wall 103. The inward and outward expansion of distal cuff 818 is illustrated in sequential FIGS. 30 and 31, with phantom lines added to more clearly show the relative movement of the cuff 818 in an expanded state depicted in FIG. 31 relative to its neutral state depicted in FIG. 30.

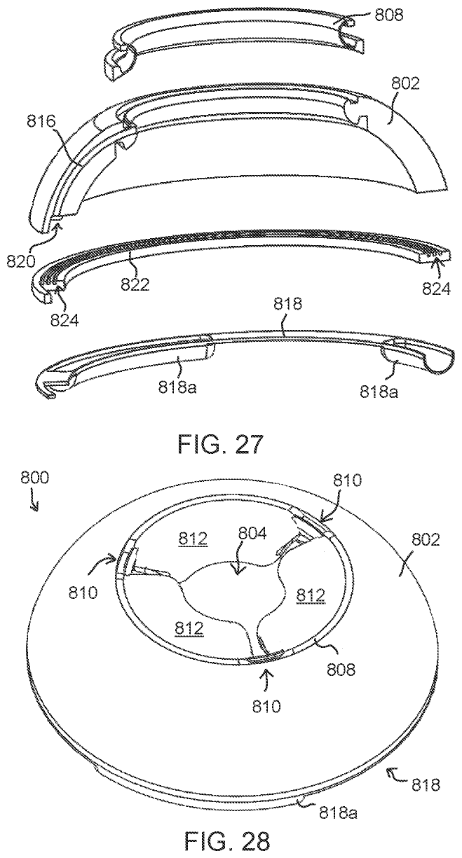

A lumen ring 822 is provided between the distal end of main body 802 and the distal cuff 818, and is in fluid connection with the inlet 820 (FIG. 27) of the fluid conduit 816 (as best shown in FIGS. 26, 27, 29, and 30). Lumen ring 822 includes open channels 824 on the proximal side of the ring. Preferably, each of the channels 824 includes at least two dedicated ports 827 open to atmosphere (FIG. 32A). With device 800 fully assembled, the channels 824 form closed lumens on the proximal side of the ring 822. The distal cuff is fitted to the bottom, distal side of the lumen ring 822, and one of the open ports 827 of each of the channels 824 is open into one of the distal cuff inflatable segments 818a. Optionally, a check valve mechanism may be provided with the device, such as in fluid connection with the fluid inlet 820, one of the channels 824, and/or an open port 827. The individual bladders 818a may each be ported to a single lumen with the other end of the lumen then ported to a check valve to independently control inflation and deflation of each bladder 818a. An inflation device, e.g. a tube, may be mated or coupled with the check valve to introduce a fluid (e.g. air or saline) to various expandable cuff elements to fill or inflate the cuffs as desired to properly fit and secure the device in the patient's vagina 103 to support the cervix 101. For example, a multi-lumen tube 828 (FIG. 32) with one-way valves, such as duck-bill check valves 830, may be provided to engage the open ports 827 to inflate the cuff components. Preferably each lumen of the tube 828 is individually addressable such that a care provider may direct inflation of only one or a selected subset of the cuff components at a time (see FIG. 33) to fine tune the positioning of the device 800 as well of the constricting force applied around the cervix 101. The ability to address each of the bladders 808 and 818a may permit the care provider to redirect or angle the cervix 101 in a particular manner to alleviate pressure and stabilize the cervix 101. For example, the provider may change the orientation of the device 800 by selectively inflating one or more of the distal cuff bladders 818a. It is contemplated that the lumens within tube 828 may provide passageways for items to be delivered to the device 800, for example, nitinol wires, springs, electrical wiring, or pharmaceuticals/medications may be passed through the tube 828 to the device 800. The tube 828 may be removably coupled to the device 800, which may provide easier movement for the patient, or the tube 828 may be permanently bonded to the device with the tube remaining inside the patient and/or extending to the exterior of the vagina 103.

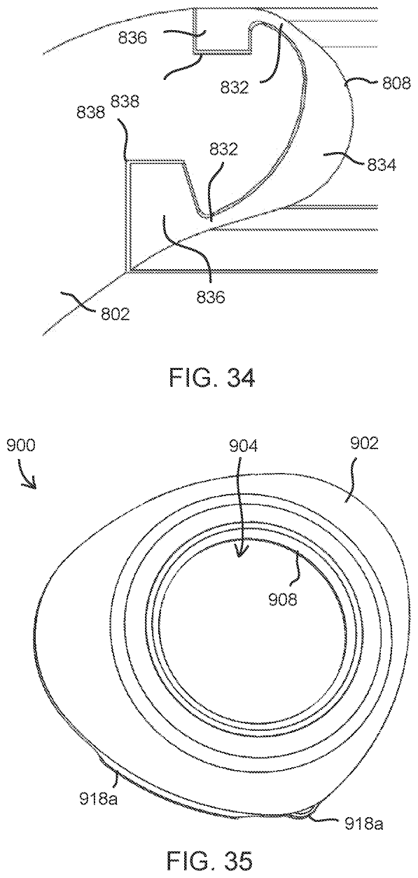

As best shown in FIG. 34, the proximal cuff 808 includes variable wall thicknesses with upper and lower thin-walled portions 832 and a thicker wall or thick-walled portion 834 at a proximal portion of the cuff 808 between the upper and lower thin-walled portions 832. The proximal cuff 808 is coupled to the proximal end of the wall of the body 802 with teeth 836 that engage grooves 838 formed in the proximal end of the wall. The thin-walled portions 832 and thick walled portion 834 function in similar fashion to portions 304 and 306 of the cuff 300 illustrated in FIG. 9 and described above.

Referring now to FIG. 35, another cervical support and reinforcement device 900 is provided for the reduction or prevention of preterm labor. The device 900 is similar to devices 100, 200, 400, 500, 700, and 900 described above, but includes some alternative and/or additional components and functions, with the more significant differences described below. Similar to the components of devices 100, 200, 400, 500, 700 and 800, device 900 includes a main body 902, a proximal opening 904, a distal opening, an expandable annular cuff 908 proximate the proximal opening 904, and an expandable annular cuff 918 proximate the distal opening, with two or more inflatable bladders 918a defining the distal cuff 918 (FIG. 35), for example (see placement example in FIGS. 1 and 2). The main body 902 of device 900 is asymmetric and non-circular or non-spherical. For example, the main body 902 includes three extended portions having larger radii and three smaller portions between the extended portions. The extended portions may provide improved engagement of the vaginal wall to counteract, limit, or prohibit rotation of the device 900 inside the vagina 103.

Referring now to the illustrative embodiment of FIGS. 36 and 37, another cervical support and reinforcement device 1000 is provided for the reduction or prevention of preterm labor. The device 1000 is similar to devices 100 and 200 described above and illustrated in FIGS. 1-6 and 7-8, but includes some alternative and/or additional components and functions. Similar to the components of devices 100 and 200, device 1000 of FIGS. 36-37 includes a main body 1002, a proximal opening 1004, a distal opening 1006, an expandable annular cuff 1008 with recessed portions 1010 and enlarged portions 1012, and a raised, annular rim 1014. As compared to devices 100 and 200, cervical support device 1000 includes an array of many ridges, cleats, or teeth 1016 arranged in a spaced arrangement along the interior perimeter of the enlarged portions 1012 of the proximal cuff 1008. The cleats 1016 are configured for gripping the exterior of the cervix 101 (see placement example in FIGS. 1 and 2) to provide additional grip to retain the cervix in an elongated and closed state and mitigate or prevent the cervix from slipping out of the proximal cuff 1008. The cleats 1016 may be formed along with the exterior wall of the cuff 1008, such as by extruding, molding, or an over-molding process, for example. It is contemplated that the wall thickness of the cuff 1008 at or adjacent to each cleat 1016 may be varied to permit the cleats 1016 to extend outwardly relative the cuff wall such that the cleat 1016 sits prouder of the cuff wall when the cuff 1008 is in the inflated state as compared to when the cuff 1008 is in the neutral state.

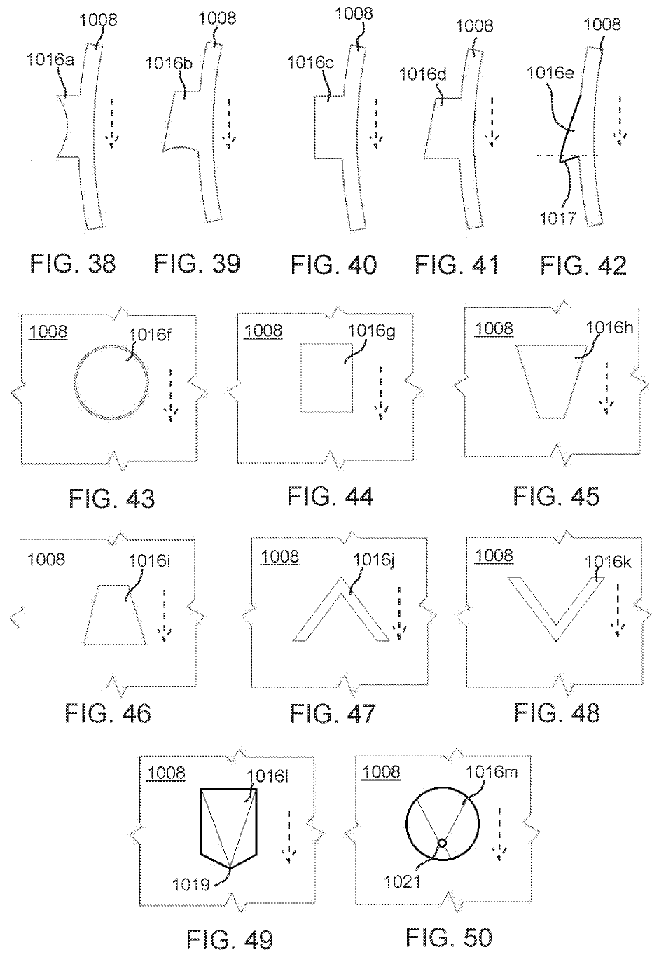

FIGS. 38-42 illustrate various exemplary shapes in which the radial profile of the cleats 1016 may be formed. For references purposes, the phantom arrow provided alongside each of the exemplary cleat profiles 1016a-1016e of FIGS. 38-42 indicates the direction toward the bottom of the cervical support device, which would therefore be the direction toward the bottom of the cervix 101 when the device is placed with the cuff 1008 encircling the cervix. In some embodiments, the cleat height (i.e. the distance that the cleat is proud of the cuff wall) may range from between about 100 micrometers (100 um) to 1500 micrometers (1500 um), for example. The cleat width (i.e. the upper to lower length of the cleat) may range from between about 100 micrometers (100 um) to 1500 micrometers (1500 um), for example. The various forms of cleat profiles 1016a-1016e of FIGS. 38-42 may be molded or formed of the same material as the cuff 1008, or the cleats may be formed of a different material or different hardness of material, such as a silicone or plastic that is more rigid than the silicone of the cuff 1008. For manufacturing purposes, it may be more preferred that the cleats are formed of the same elastic material as the cuff 1008. It is contemplated that the cleats 1016 may be individually configured to act or function as suction cups to form a vacuum engagement with the tissue of the exterior of the cervix 101. As illustrated in FIGS. 38-42, the cleat profiles 1016a, 1016b, 1016c, 1016d, and 1016e are illustrated as solid bodies. However, it will be appreciated that the cleats may be formed with voids between their perimeters and the cuff wall, or the cleats may be formed with voids that open into the cuff 1008 such that as the cuff is inflated, the voids in the cleats may be inflated or expanded to expand the cleats. As illustrated in FIG. 42, the cleat 1016e may be formed such that the downward edge/tip 1017 forms an acute angle with the exterior wall of the cuff 1008. This configuration may effectively function as a hook to grab and hold the cervical tissue and thus provide improved engagement with the cervical tissue.

FIGS. 43-50 illustrate various exemplary shapes in which the front, elevation shape of the cleats 1016 may be formed. For references purposes, the phantom arrow provided with each of the exemplary cleat shapes 1016f-1016m indicates the direction toward the bottom of the cervical support device, which would therefore be the direction toward the bottom of the cervix 101 when the device is placed with the cuff 1008 encircling the cervix. Any of the cleat shapes 1016f-1016m (in addition to other suitable cleat shapes) may be utilized with any of the cleat profiles 1016a-1016e (in addition to other suitable cleat profiles) to form a desired cleat 1016 for engaging the cervix 101. The cleat shape 1016f depicted in FIG. 43 is circular and may have a diameter that ranges between about 50 micrometers (50 um) to 2000 micrometers (2000 um), for example. FIGS. 44-49 illustrate non-circular cleat shapes 1016g-1016l, which may have overall height and width dimensions that range between 25 micrometers (25 um) to 2500 micrometers (2500 um), for example. FIGS. 49 and 50 depict additional geometric cleat shapes 1016l and 1016m that each extend outward from the cuff wall 1008 to a pointed end or tip 1019 and 1021, respectively. Cleat shape 1016l of FIG. 49 is a generally pyramidal shape, and in the illustrated embodiment is an oblique pyramid with the tip 1019 offset toward its lower side. Cleat shape 1016m is a generally conical shape, and in the illustrated embodiment is an oblique cone with the tip 1019 offset toward its lower side.

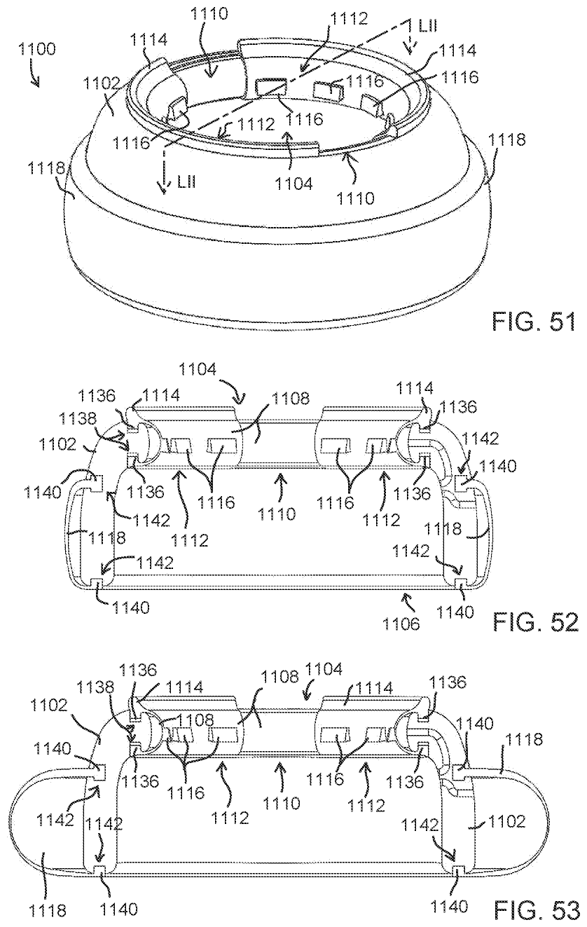

Referring now to the illustrative embodiment of FIGS. 51-54, another cervical support and reinforcement device 1100 is provided for the reduction or prevention of preterm labor. The device 1100 is similar to devices 100, 500, and 1000 described above and illustrated in FIGS. 1-6, 19-20, and 36-37, but includes some alternative and/or additional components and functions. Similar to the components of devices 100, 500, and 1000 described above, device 1100 of FIGS. 51-54 includes a main body 1102, a proximal opening 1104, a distal opening 1106, an expandable annular proximal cuff 1108 with recessed portions 1110 and enlarged portions 1112, and a raised, annular rim 1114. In further similarity to the components of device 500, device 1100 includes a second expandable annular cuff 1118 proximate the distal opening 1106 and/or exterior of body 1102. The second cuff 1118 is hereinafter referred to as “distal cuff” 1118. In further similarity to the components of device 1000, device 1100 includes an array of cleats or teeth 1116 arranged in spaced arrangement around an interior perimeter of the enlarged portions 1112 of the proximal cuff 1108. As compared to device 500, the distal cuff 1118 of cervical support device 1100 is relatively larger than cuff 518 and extends further up the wall of the body 1102 of the device 1100 than that of cuff 518. Exemplary embodiments of cuff 1118 may extend halfway up the exterior wall of the body 1102, or substantially the entire length of the exterior wall of the body 1102, for example. The increased size increases the contact area between the cuff 1118 and the vaginal wall 103 to provide improved support to the cervix 101 and/or uterus 105. It will be appreciated that distal cuff 1108 may be formed with a single bladder, or with two or more bladders similar to bladders 818a of distal cuff 818 described above and illustrated in FIGS. 25-31. Further, as compared to device 1000, the cleats 1116 of cervical support device 1100 are significantly larger than cleats 1016 of device 1000, and fewer cleats 1116 are provided around the interior perimeter of the enlarged portions 1112. The cleats 1116, like cleats 1016, are configured for gripping the exterior of the cervix 101 (see placement example in FIGS. 1 and 2) to attempt to provide additional grip to retain the cervix in an elongated and closed state and mitigate or prevent the cervix from slipping out of the proximal cuff 1108. The cleats 1116 may be formed along with the exterior wall of the cuff 1108, such as by extruding, molding, or an over-molding process, for example. It is contemplated that the wall thickness of the cuff 1108 at or adjacent to each cleat 1116 may be varied to permit the cleats 1116 to extend outwardly relative the cuff wall such that the cleats 1116 sit prouder to the cuff wall when the cuff 1108 is in the inflated state as compared to when the cuff 1108 is in the neutral state.

Similar to the teeth 836 and groove 838 arrangement of the proximal cuff 808 and wall of body 802 of device 800 described above and illustrated in FIGS. 25-31, the proximal cuff 1108 of device 1100 is coupled to the proximal end of the wall of the body 1102 with teeth 1136 that engage grooves 1138 formed in the proximal end of the wall (FIGS. 52-54). The distal cuff 1118 of device 1100 is coupled at one end to the distal end of the wall of body 1102 (e.g. at the distal most portion of the exterior of the wall) and at the opposite end to a central exterior portion of the wall of the body 1102. The respective ends of the distal cuff 1118 are coupled to the body 1102 with teeth 1140 that engage a corresponding groove 1142 formed in the wall of the body 1102.

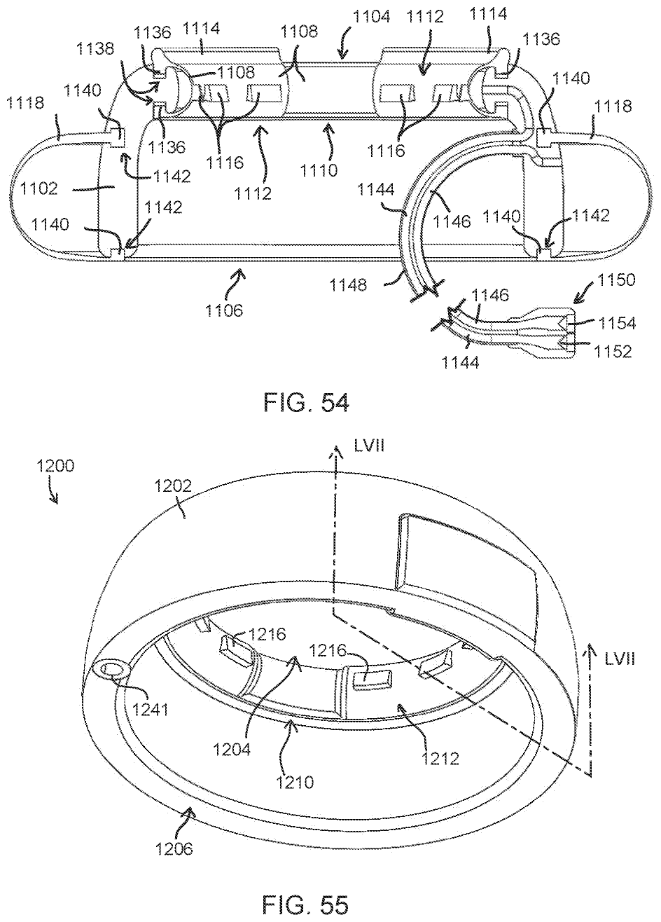

Device 1100 includes fluid conduits 1144 and 1146 which are in fluid communication with the proximal cuff 1108 and the distal cuff 1118, respectively (FIG. 54). The fluid conduits 1144 and 1146 are similar in structure and function to conduits 116, 216, 516, and 816 of respective devices 100, 200, 500, and 800 (FIGS. 5 and 6, 7 and 8, 19 and 20, and 29 and 30, respectively). As shown in FIG. 54, the conduits 1144 and 1146 are dedicated to corresponding cuffs 1108 and 1118 and extend from the body 1102 of device 1100 inside a tube 1148 that extends from the body 1102 toward the interior of the device 1100. A valve system 1150, which is similar in function to valve system 826 described above and illustrated in FIGS. 29-30, is provided at a distal end of the tube 1148, and provides a valve 1152 and 1154 for each conduit 1144 and 1146, such as one-way duckbill valves, for example. The tube 1148 may have a length sufficient to extend from the device (when placed in the patient's vagina) and out through the vaginal opening such that a care provider may easily access the valve system 1150 without removing or repositioning the device within the patient. An advantage to having the valve exposed to the exterior of the vaginal opening is that a care provider may simply break a pressure seal to release the pressure in the conduits 1144 and 1146 and cuffs 1108 and 1118, which would otherwise be trapped due to the function of the one-way valves 1152 and 1154. For example, a commonly known or commercially available device may be utilized to permit the care provided to readily break the pressure seal, such as a locking needle or syringe tip coupled with the tube 1148. The care provider may break the pressure seal by removing the valve system 1150 from the tube 1148.

Referring now to the illustrative embodiment of FIGS. 55-57, another cervical support and reinforcement device 1200 is provided for the reduction or prevention of preterm labor. The device 1200 is similar to device 1100 described above and illustrated in FIGS. 51-54, but includes some alternative and/or additional components and functions. Similar to the components of device 1200 described above, device 1200 of FIGS. 55-57 includes a main body 1202, a proximal opening 1204, a distal opening 1206, an expandable annular proximal cuff 1208 with recessed portions 1210, an array of cleats or teeth 1216 arranged in spaced arrangement around an interior perimeter of the enlarged portions 1212 of the proximal cuff 1208, and teeth 1236 that engage grooves 1238 formed in the proximal end of the wall (FIG. 57). Unlike device 1100, device 1200 does not include an expandable annular distal or exterior cuff, however, it will be appreciated that a distal or exterior cuff may be provided with device 1200, if desired.

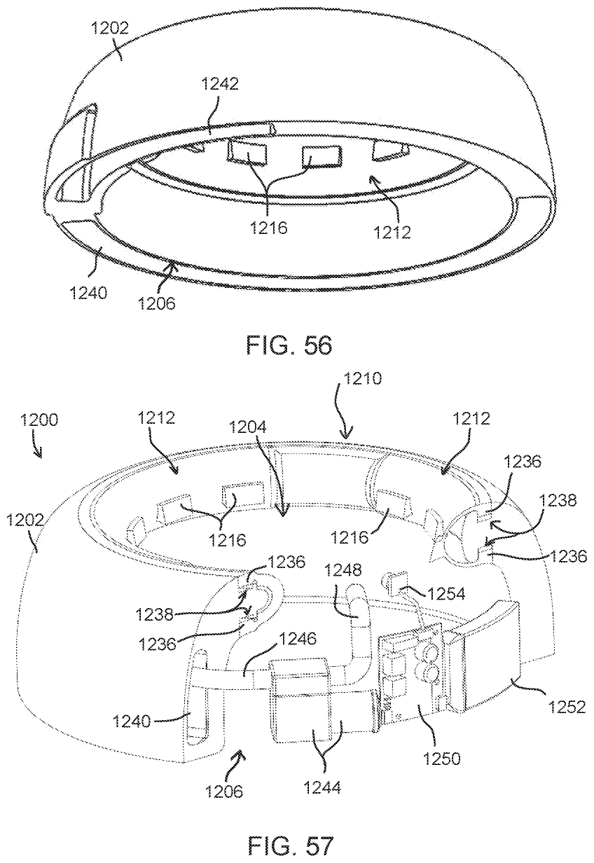

A fluid reservoir 1240 is formed inside a wall of the body 1202 and in fluid communication with the cuff 1208 (FIGS. 56 and 57). Device 1200 includes a valve system with an inlet port 1241 (FIG. 55). The inlet port 1241 may be formed as a one-way valve, such as described previously for devices 800 and 1100, for example. Device 1200 includes a component cavity 1242 formed inside another portion of the wall of the body 1202, adjacent to the fluid reservoir 1240 (FIG. 56). The component cavity 1242 is provided for housing an internal fluid pump 1244 that is positioned inside the wall of the body 1202 and in fluid communication with fluid reservoir 1240 via a first conduit 1246 and in fluid communication with the cuff 1208 via a second conduit 1248 (FIG. 57). The pump 1244 is operable to move fluid from the fluid reservoir 1240 to one or more portions of the cuff 1208 to inflate and/or deflate the cuff. The inlet port 1241 may be configured as a vent to enable air transfer to counteract a vacuum forming during operation of the pump 1244.

A controller or computer 1250 is electronically coupled with the pump 1244 to control operation of the pump 1244. A power source, in the form of a rechargeable battery 1252, is coupled with the pump 1244 and the controller 1250. The controller 1250 may be enabled for wireless communication/control such that a care provider may remotely inflate/deflate the cuff 1208 without any invasive procedure to access the device 1200, thus enabling the cervical support device 1200 to be a closed system, requiring no physical manipulation to adjust the device. For example, the controller may include a Bluetooth® wireless communication or other remote control device. A charging cable for charging the power source may be provided for charging the battery 1252, either internally or externally of the patient. A pressure transducer 1254 is coupled with the controller 1250 to supply the care provider with information regarding the status of the device 1200, such as the state of inflation/deflation of the cuff 1208 and the force being exerted on the cervix 101 or vagina 103. In some embodiments, the pressure transducer 1254 may be adapted to detect a drop in pressure within acceptable bounds (as set by the care provider), and the pump 1244 may be configured to automatically add fluid to the under pressurized cuff to maintain the desired level of inflation. In an optional embodiment, a position sensing device (e.g. a solid state gyroscopic module) may be provided to detect orientation data of the cervical support device 1200 (e.g. the solid state gyroscopic module may detect the relative angle of the device relative to the gravity vector). Based on the position data, the device may be adapted to automatically adjust the angle of the device in the event that the device shifts beyond a desired position (as set by the care provider).

It will be appreciated that the various different components and functions of the embodiments of the cervical support devices described herein and illustrated in FIGS. 1-57, may be adapted for use with any of the other embodiments disclosed herein.

Thus, the cervical support devices of the various illustrative embodiments detailed above provide selectively adjustable devices for supporting an insufficient cervix to promote extended gestation of pre-term pregnancies. The cervical support devices include an upper or proximal cuff for supporting and/or constricting a patient's cervix in an attempt to impede the cervix from opening or further shortening. The proximal cuff may include multiple expandable bladders which may be independently addressable (i.e. inflatable or deflatable) to provide customizable support for the cervix based on the specific needs of the patient. In a preferred embodiment, the proximal cuff provides a transverse force as well as a downward force (i.e. away from the uterus) to the cervix, which may promote retention of the cervix within the proximal cuff. A lower or distal cuff may be provided to seat or secure the body of the device on the patient's vaginal wall. The distal cuff may also include multiple expandable bladders that may be independently addressable to provide customizable support for the device. Preferably, the proximal cuff is shaped and dimensioned to permit blood flow from the upper cervix to the lower cervix protruding through the proximal cuff.

Changes and modifications in the specifically described embodiments can be carried out without departing from the principles of the present invention, which is intended to be limited only by the scope of the appended claims, as interpreted according to the principles of patent law including the doctrine of equivalents.

1. A cervical support and reinforcement device comprising:
a body defining an interior passageway for receiving a cervix, said body comprising upper and lower portions defining proximal and distal openings to said interior passageway, respectively, and an exterior surface for engaging a vaginal wall; and
a cuff coupled to said body at said proximal opening and configured to surround at least a portion of the cervix, said cuff comprising an expandable portion adjacent a recessed portion, wherein said expandable portion is selectively extendable further into said interior passageway than said recessed portion.

2. The cervical support and reinforcement device of claim 1, wherein said body comprises a semi-spheroid shape and said proximal opening defines a smaller area than said distal opening.

3. The cervical support and reinforcement device of claim 1, further comprising a ridge disposed along an interior perimeter of said cuff for gripping an exterior of the cervix.

4. The cervical support and reinforcement device of claim 1, wherein said cuff, upon expansion, is configured to expand substantially radially inward toward the axial center of said proximal opening.

5. The cervical support and reinforcement device of claim 1, wherein said cuff, upon expansion, is configured to expand both radially inward and downward away from the transverse plane of said proximal opening and away from a uterus as said cuff expands around the cervix.

6. The cervical support and reinforcement device of claim 5, wherein said cuff comprises a non-uniform wall thickness.

7. The cervical support and reinforcement device of claim 5, wherein said cuff comprises a plurality of inflatable bladders adjacent and co-axial with one another.

8. The cervical support and reinforcement device of claim 1, further comprising a rim disposed at the upper portion of said body and extending upward and away from said proximal opening, said rim configured to guide the cervix into said proximal opening when said device is being placed.

9. The cervical support and reinforcement device of claim 1, further comprising a second expandable cuff disposed at one or both chosen from the exterior portion of said body and the lower portion of said body proximate said distal opening, said second cuff expandable away from the axial center of said distal opening to expand toward and into the vaginal wall, said second cuff expandable from a neutral state to an expanded state in which an exterior perimeter of said second cuff is enlarged as compared to its exterior perimeter when in the neutral state.

10. The cervical support and reinforcement device of claim 1, further comprising a fluid conduit in fluid communication with said cuff, and a valve in fluid communication with said fluid conduit opposite said cuff.

11. The cervical support and reinforcement device of claim 10, wherein said valve is operable to removably receive a portion of an inflation device operable to inflate said cuff without removal of said device from a patient's vagina.

12. The cervical support and reinforcement device of claim 10, further comprising a second expandable cuff disposed at one or both chosen from the exterior portion of said body and the lower portion of said body proximate said distal opening, said second cuff expandable away from the axial center of said distal opening to expand toward and into the vaginal wall, wherein said second cuff is in fluid communication with said fluid conduit.

13. The cervical support and reinforcement device of claim 1, wherein said cuff comprises at least two of said recessed portions and at least two of said expandable portions alternatingly spaced with said recessed portions.

14. The cervical support and reinforcement device of claim 1, further comprising a pump and a fluid reservoir disposed in said body, said pump in fluid communication with said fluid reservoir and said fluid reservoir in fluid communication with said cuff, wherein said pump is adapted to move fluid between said fluid reservoir and said cuff for inflation and deflation of said cuff.

15. The cervical support and reinforcement device of claim 14, further comprising a wireless control device for remotely controlling said pump.

16. A cervical support and reinforcement device for placement in a vagina, said device comprising:
a body defining an interior passageway for receiving a cervix, said body comprising upper and lower portions defining proximal and distal openings to said interior passageway, respectively, and an exterior surface for engaging a vaginal wall to support said device proximate the cervix; and
a cuff coupled to said body at said proximal opening and configured to surround a least a portion of the cervix, said cuff expandable from a neutral state to an expanded state in which said cuff is extended both radially inward and downward away from the transverse plane of said proximal opening and away from a uterus.

17. The cervical support and reinforcement device of claim 16, wherein said cuff comprises one or more expandable portions adjacent one or more recessed portions, wherein said expandable portions are selectively extendable further into said interior passageway than said recessed portions.

18. The cervical support and reinforcement device of claim 16, wherein said cuff comprises a non-uniform wall thickness.

19. The cervical support and reinforcement device of claim 16, wherein said cuff comprises a plurality of inflatable bladders adjacent and co-axial with one another.

20. The cervical support and reinforcement device of claim 16, further comprising a rim disposed at the upper portion of said body and extending upward and away from said proximal opening, said rim configured to guide the cervix into said proximal opening when said device is being placed.

21. The cervical support and reinforcement device of claim 16, further comprising an expandable distal cuff disposed at the lower portion of said body proximate said distal opening, said distal cuff expandable away from the axial center of said distal opening to expand toward and into the vaginal wall or fornix.

22. A cervical support device for placement in a vagina, said device comprising:
a semi-spheroid body defining an interior passageway for receiving a cervix, said body comprising upper and lower portions defining proximal and distal openings, respectively, and an exterior surface for engaging a vaginal wall to support said device proximate the cervix; and
a cuff coupled to said body at said proximal opening and configured to surround at least a portion of the cervix, said cuff comprising:
a first bladder that is coaxial with said proximal opening; and
a second bladder that is coaxial with said first bladder and said proximal opening;
wherein said first bladder and said second bladder are selectively inflatable and extendable into said interior passageway.

23. The cervical support device of claim 22, wherein said cuff is expandable from a neutral state to an expanded state in which said cuff is expanded both radially inward toward the axial center of said proximal opening, and downward away from the transverse plane of said proximal opening and away from a uterus.
